(19) United States
(12) Patent Application Publication
(10) Pub. No.: US 2025/0177505 A1
(43) Pub. Date: Jun. 5, 2025

(54) CANCER VACCINE COMPRISING EPITOPE OF C-MET AND EPITOPE OF HIF1ALPHA, AND USE THEREOF

(71) Applicant: KOREA UNIVERSITY RESEARCH AND BUSINESS FOUNDATION, Seoul (KR)

(72) Inventor: Kyong Hwa PARK, Seoul (KR)

(21) Appl. No.: 18/836,943

(22) PCT Filed: Feb. 7, 2023
(86) PCT No.: PCT/KR2023/001715

§ 371 Date: Aug. 8, 2024

(30) Foreign Application Priority Data
Feb. 8, 2022 (KR) 10-2022-0016462
Aug. 17, 2022 (KR) 10-2022-0102718
Feb. 3, 2023 (KR) 10-2023-0015177

(51) Int. Cl. A61K 39/00; A61P 35/04; C07K 16/28

(52) U.S. Cl. CPC A61K 39/00111; A61P 35/04; C07K 16/2818; A61K 2039/505

(57) ABSTRACT

The present invention relates to: a use of an epitope of c-MET and an epitope of HIF1α as a cancer vaccine; and a use of said cancer vaccine. The cancer vaccine according to the present invention decreases the expression level of HIF1α and/or c-MET and the expression of angiogenesis-related markers in various types of cancer known to overexpress the protein, and can inhibit tumor growth and furthermore, activates T1 cells and induces infiltration of the tumor, and thus can be used as a general-purpose cancer vaccine for suppressing the progression of cancer and preventing the metastasis thereof without being limited to the above-mentioned cancer types. In addition, the vaccine according to the present disclosure can more effectively inhibit the growth and progression of cancer and the formation of metastatic cancer by being administered in combination with an immune checkpoint inhibitor.

### TECHNICAL FIELD

The present disclosure relates to a use of an epitope of c-MET and an epitope of HIF1α as a cancer vaccine, and a use of the cancer vaccine.

### BACKGROUND ART

Breast cancer is the most commonly diagnosed cancer in women worldwide and the leading cause of cancer-related deaths. Triple negative breast cancer (TNBC) has no three receptors commonly found in breast cancer, that is, an estrogen receptor, a progesterone receptor, and a human epidermal growth factor receptor 2 (HER2). The TNBC accounts for 10 to 15% of the entire breast cancer. In particular, the TNBC is the most aggressive cancer type of breast cancer and has more aggressive biological characteristics than other breast cancer subtypes. However, except for recent progress with a PIK3CA/Akt pathway inhibitor which is currently in phase 3 clinical trials, the treatment target for TNBC is unclear. In addition, PIK3CA/Akt mutations are promising targets and are currently in phase 3 clinical trials. Novel treatment strategies are needed because disease-free survival and overall survival periods are significantly shortened due to lack of sensitivity to drugs and limited targeted treatment options. The TNBC is a unique subtype of metastatic cancer for which the survival period is prolonged in some patients when combined with immunotherapy. Accordingly, it is worth searching more therapies that stimulate the immune system to overcome the clinical problems of TNBC treatment.

It is estimated that 20 to 30% of early breast cancer develop into metastatic cancer. In all breast cancer patients, the bone is considered the most common site of advanced breast cancer, and an incidence rate is close to 80%. In general, patients with bone metastasis tend to survive longer and have better clinical outcomes than patients with visceral metastasis. However, patients with TNBC bone metastasis have a poor prognosis, similarly to patients with visceral metastasis. Therefore, the occurrence of bone metastasis in TNBC patients is one of the major clinical challenges in patients with advanced cancer. A better understanding of the mechanisms based on spread and colonization into bone of breast cancer cells is expected to provide new insights into the development of targeted therapies.

Hypoxia in tumors is known to be associated with cancer progression and poor clinical outcomes in breast cancer patients. The TNBC often exhibits morphologic features of hypoxia, such as the presence of fibrous and necrotic areas. In particular, high expression of HIF-1α negatively affects the survival of TNBC. In addition, many genes involved in resistance, proliferation, invasion and metastasis, immune evasion, as well as angiogenesis, cell survival, chemotherapy and radiation are known to be regulated through HIF-1α. In previous studies, the importance of HIF-1α was demonstrated using MDA-MB-231 cells in an immunodeficient mouse model. It has been reported that in the bone metastasis process, HIF-1α signaling contributes to bone metastasis by inhibiting osteoblast differentiation and promoting osteoclastogenesis to regulate the bone microenvironment. In previous studies of patients with TNBC, tumors overexpressing the HIF-1α protein did not survive longer than tumors with low expression of the HIF-1α protein. In addition, it has been reported that abnormal overexpression of self-proteins enhances immunogenicity. Considering that the expression of HIF-1α is specifically increased in TNBC, targeting HIF-1α may provide a novel therapeutic option for TNBC patients.

The c-MET has been reported to be overexpressed in approximately 52% of TNBC and is associated with reduced disease-free and overall survival rates. C-MET, which is primarily expressed in epithelial cells, induces various intracellular signaling pathways required for the development and progression of many human cancers. In previous studies, it has been reported that a hepatocyte growth factor (HGF)/MET mechanism acts as an important mediator between epithelial breast cancer cells and mesenchymal cells in the bone microenvironment to contribute to the progression of osteolytic bone metastasis in vivo. Accordingly, the role and regulation of HGF/MET in the metastatic bone microenvironment requires additional investigation.

It is important to understand the tumor microenvironment because malignant tumor of breast cancer is influenced not only by genetic abnormalities and biological characteristics, but also by the interaction between cancer cells and the microenvironment. The tumor microenvironment of breast cancer includes diverse cell types and tumor-infiltrating lymphocytes (TILs). According to analysis of randomized clinical trials, it has been reported that a high level of TILs is associated with a reduced risk of recurrence and death, and that TILs are more abundant in TNBC compared to other breast cancer subtypes. Studies have shown that various TIL subtypes participate in breast cancer immune responses and cross-acceptance of subgroup cells to commonly mediate and regulate tumor-associated immunity. Tumor-infiltrating type I T cells, particularly CD8 T cells, are the lymphocytes most commonly associated with TNBC survival. In addition, high levels of tumor-infiltrating CD8 T cells have been known to be associated with improved responses to chemotherapy.

Treating cancer by regulating the immune system is called cancer immunotherapy, and various approaches have been developed to fight cancer by activating the immune system of cancer patients. In particular, various tumor-associated antigens (TAAs) or neoantigens expressed in cancer cells may induce anti-tumor immune responses and may be used as immunotherapy such as cancer vaccines. Currently, many cancer vaccine platforms have been developed, including peptide or protein-based vaccines, oncolytic virus or recombinant viral vector vaccines, dendritic cell vaccines, and engineered cell vaccines. In addition, recent advances in effective immunological adjuvants may enhance the utility of vaccines for various therapeutic purposes.

A cancer vaccine based on a peptide based on a Major Histocompatibility Complex (MHC) class II epitope induces tumor-specific T cell responses using a TAA-derived tumor-specific peptide, and is in clinical trials as an attractive cancer therapeutic agent due to its low toxicity. A peptide vaccine based on an MHC class II peptide (13 to 25 amino acids) is longer than an MHC class I peptide (8 to 10 amino acids) and primarily stimulates CD4 T cells. Vaccination with an MHC class I-restricted peptide may induce immune tolerance by not inducing sufficient cytotoxic T-lymphocytes (CTLs). However, the MHC class II restricted peptide has an advantage of being able to sufficiently induce CTLs and T-helper cells. In addition, there are studies that peptide vaccines based on IGF-IR, IGFBP-2, HER2, or HIF-1α class II epitopes may induce both T-helper cells and CTLs. Evidence continues to accumulate that the MHC class II-based vaccine has strengths in generating immune memory and epitope spreading.

Tumor cells may evade immune surveillance through immune checkpoint pathways such as Tim-3, PD-1, LAG-3, and CTLA-4. Immune checkpoint inhibitors (ICIs) block these pathways and improve anti-tumor immune responses in a variety of tumor types, including melanoma, lung, renal cell, and bladder cancers. However, in patients with other malignant tumors, such as breast cancer, prostate cancer, and pancreatic cancer, the effect of a single immune checkpoint inhibitor was rarely observed.

Peptide-based cancer vaccines have been shown to have fewer side effects and better survival than conventional treatments, but the peptide-based cancer vaccines alone are considered insufficient to maintain cancer control. In previously reported animal experiments, combination therapy with the immune checkpoint inhibitors and various types of cancer vaccines increased tumor-specific T cell activation and antitumor effects. Combination therapy of peptide-based cancer vaccines and immune checkpoint inhibitors to activate tumor-specific immune responses and deactivate immunosuppression in the tumor microenvironment may induce stronger anti-tumor responses.

### DISCLOSURE OF THE INVENTION

### Technical Goals

An aspect to be achieved by the present disclosure is to provide a method for preventing or treating cancer by inducing an immune response with a HIF-1α epitope and a c-Met epitope, and to provide a use of the two epitopes as a cancer vaccine.

Further, another aspect to be achieved by the present disclosure is to provide a combined administration method of the vaccine and the immune checkpoint inhibitor for effective prevention or treatment of cancer.

However, technical goals to be achieved are not limited to those described above, and other goals not mentioned above are clearly understood by one of ordinary skill in the art from the following description.

### Technical Solutions

In order to solve the aspect, the present disclosure provides a use of an epitope peptide of HIF1α and an epitope peptide of c-MET as a cancer vaccine.

Therefore, the present disclosure provides a pharmaceutical composition for preventing or treating cancer, including an epitope of HIF1α and an epitope of c-MET as an active ingredient.

As an embodiment of the present disclosure, the two epitopes may be provided as separate peptides and may also be provided as one polypeptide.

As another embodiment of the present disclosure, the epitope peptide of c-Met may include at least one amino acid sequence selected from the group consisting of amino acid sequences represented by SEQ ID NOs: 1 and 2.

As another embodiment of the present disclosure, the epitope peptide of c-Met may include or consist of an amino acid sequence represented by SEQ ID NO: 1 and include or consist of an amino acid sequence represented by SEQ ID NO: 2.

As another embodiment of the present disclosure, the epitope peptide of c-Met may include or consist of amino acid sequences represented by SEQ ID NOs: 1 and 2.

As another embodiment of the present disclosure, the epitope peptide of HIF1α may include at least one amino acid sequence selected from the group consisting of amino acid sequences represented by SEQ ID NOs: 3 to 5.

As another embodiment of the present disclosure, the epitope peptide of HIF1α may include or consist of an amino acid sequence represented by SEQ ID NO: 3, include or consist of an amino acid sequence represented by SEQ ID NO: 4, and include or consist of an amino acid sequence represented by SEQ ID NO: 5.

As another embodiment of the present disclosure, the epitope peptide of HIF1α may include or consist of amino acid sequences represented by SEQ ID NOs: 3 and 4, include or consist of amino acid sequences represented by SEQ ID NOs: 4 and 5, and include or consist of amino acid sequences represented by SEQ ID NOs: 3 and 5.

As another embodiment of the present disclosure, the epitope peptide of HIF1α may include or consist of amino acid sequences represented by SEQ ID NOs: 3 to 5.

As another embodiment of the present disclosure, when the epitope peptide of HIF1α and the epitope peptide of c-MET each includes two or more sequences in the sequence list provided by the present specification, one to three amino acids may be further included at a level that does not affect the structure formed by the amino acid sequence of each sequence number, that is, does not affect the function of a secondary structure of each amino acid sequence, and the amino acid sequences of each sequence number may be directly linked or may be linked by a linker that does not affect the structure and function thereof.

As another embodiment of the present disclosure, when the two epitopes are provided as one polypeptide, the polypeptide may include or consist of an amino acid sequence represented by SEQ ID NO: 6.

As another embodiment of the present disclosure, the pharmaceutical composition may be administered in combination with an immune checkpoint inhibitor. At this time, the combined administration means simultaneous or sequential administration with the immune checkpoint inhibitor, and the simultaneous administration means administration of other drugs within 24 hours after the pharmaceutical composition or the immune checkpoint inhibitor is administered to a subject, and is not necessarily limited to the meaning that two drugs are administered to a subject as one composition. During the sequentially combined administration, the order is not limited, but desirably, the immune checkpoint inhibitor may be administered after administering the pharmaceutical composition.

As another embodiment of the present disclosure, the pharmaceutical composition may further include an immune checkpoint inhibitor.

As another embodiment of the present disclosure, the immune checkpoint inhibitor may block signaling of one or more immune checkpoint proteins selected from the group consisting of CTLA-4, PD-1, PD-L1, PD-L2, LAG-3, BTLA, B7H3, B7H4, TIM3, KIR, TIGIT, CD47, VISTA and A2aR, and specifically an antibody targeting the one or more of immune checkpoint proteins, and the antibody may be a monoclonal antibody.

As another embodiment of the present disclosure, the cancer is not limited to any cancer type in which c-MET and/or HIF1α overexpression has been reported, but may be desirably at least one cancer type selected from the group consisting of breast cancer, non-small cell lung cancer, stomach cancer, head and neck cancer, kidney cancer, liver cancer, prostate cancer, and thyroid cancer, and the breast cancer may include triple-negative breast cancer.

As another embodiment of the present disclosure, the cancer includes advanced cancer and/or metastatic cancer, and the metastatic cancer means systemic metastatic cancer, and includes metastatic bone tumor.

Further, the present disclosure provides a method for preventing or treating cancer, including administering an epitope of HIF1α and an epitope of c-MET to a subject.

As an embodiment of the present disclosure, the subject is not limited to a mammal in need of prevention or treatment of cancer, and desirably, may be a human developed with cancer and at risk of metastasis.

As another embodiment of the present disclosure, the method may further include administering an immune checkpoint inhibitor to the subject.

Further, the present disclosure provides a use of an epitope of HIF1α and an epitope of c-MET for the preparation of a drug for the prevention or treatment of cancer.

### Effects of the Invention

According to the present disclosure, a HIF1a/c-MET polypeptide cancer vaccine can decrease the expression level of HIF1α and/or c-MET and the expression of angiogenesis-related markers in various types of cancer known to overexpress the protein, and inhibit tumor growth. Furthermore, the cancer vaccine induces antigen-specific T1 cells and increases tumor infiltration, and thus can be used as a general-purpose prevention or treatment of cancer without being limited to the cancer types, and particularly, is expected to be used as a vaccine for suppressing the progression of cancer and preventing the metastasis thereof. In addition, the cancer vaccine of the present disclosure can be used as a therapy administered in combination with an immune checkpoint inhibitor.

### BRIEF DESCRIPTION OF DRAWINGS

A of FIG. 1 shows representative images for each time point, as results of confirming tumor growth and spread through bioluminescence at 14, 21, 28, and 35 days after intracardiac injection of M6-bone cells into C3(1)-Tag mice. B of FIG. 1 shows representative images of radiograph taken of the hind limb using an X-ray film 35 days after inoculation of M6-bone cells to confirm osteolytic lesions, which may confirm typical sides of soluble lesions (white arrows) observed in C3(1)-Tag mice inoculated with tibia-stable cells compared with a control. C of FIG. 1 shows results of confirming the presence of tumor cells by sacrificing mice after 35 days and performing H&E staining of paraffin sections of metastatic liver, lung, and bone (scale bar=50 μm).

FIG. 2 shows results of confirming the expression levels of c-MET and HIF1α in metastatic bone tumors using RNA sequencing and IHC analysis. A of FIG. 2 illustrates a heatmap of hierarchical clustering showing differentially expressed genes (rows) between M6 cells, M6-bone cells, and three metastatic bones (fold-change>2, p<0.05). Yellow indicates upregulation and blue indicates downregulation. B of FIG. 2 illustrates results of immunohistochemical staining for HIF1α and c-MET in metastatic bone, liver, and lung samples (scale bar=50 μm).

FIG. 3 shows results of immune microenvironment evaluation in metastatic bone tumors. Data shown for a T cell panel represent the distribution of tumor cells (blue) in CD8 T cells (red), CD4 T cells (yellow), and MDSC (green). Immune cell infiltration in normal and metastatic bones is expressed as mean±SEM (*, p<0.05).

FIG. 4 shows results of measuring the tumor volume after subcutaneously injecting 50 μg of HIF1a/c-MET polypeptide vaccine into C3-Tag mice three times at 10-day intervals and subcutaneously injecting 5×10⁵ cells/100 μl of M cells on day 10 after the final injection. The mean tumor volumes of a control group and an HIF1α/c-MET vaccine group were expressed as mean±SEM (*, p<0.05).

FIG. 5 shows results of measuring mean IFN-γ spot per well of mice immunized with HIF1α and c-MET MHC class II epitopes and confirming that the HIF1α/c-MET polypeptide vaccine promotes IFN-γ secretion of Th1 cells. Experimental groups were divided into unstimulated cells and cells stimulated with TT as a negative control or HIF1α and c-MET peptides (***p<0.001).

FIG. 6 shows results of evaluating the degree of infiltration of immune cells in implanted tumors. Data shown for a T cell panel represent the distribution of tumor cells (blue) in CD8 T cells (red), CD4 T cells (yellow), and MDSC (green). Immune cell infiltration in normal and metastatic tumor is expressed as mean±SEM (p=ns).

FIG. 7 shows results of immunofluorescence staining for HIF1α and c-MET in metastatic tumors. Green represents HIF1α and red represents c-MET (scale bar=50 μm).

FIG. 8 shows results of confirming that the HIF1α/c-MET polypeptide vaccine inhibits bone metastasis. Particularly, FIG. 8A illustrates results of tracking tumor growth and spread through luciferase imaging at 14, 21, 28, and 35 days after M6-bone cells are injected intracardiacly into mice immunized with CFA/IFA or HIF1α/c-MET. FIG. 8B shows a graph of quantifying the growth of metastatic bone tumors in the bones of mice immunized with CFA/IFA or HIF1α/c-MET over time. FIG. 8C shows micro-CT images (indicated by arrows) of mouse bones immunized with CFA/IFA or HIF1α/c-MET.

FIG. 9 shows results of confirming CD4 and CD8 infiltration in metastatic bone tumors through immunofluorescence staining. CD8 is indicated in green and CD4 is indicated in red (scale bar=20 μm). CD4 and CD8 expression in normal and metastatic bone tumors is expressed as mean±SEM (*, p<0.05; ***, p<0.001).

FIG. 10 shows representative H&E and TRAP staining images of mouse bones immunized with CFA/IFA or HIF1α/c-MET (indicated by arrows), which confirm that the formation of TRAP-positive osteoclasts is reduced by HIF1α/c-MET polypeptide vaccine administration.

FIG. 11 shows images of confirming the formation of osteoclasts by treating BMM with M-CSF (30 ng/mL), RANKL (100 ng/mL), and IFN-γ (50, 100 ng/ml) for 1, 3, 5, and 7 days (indicated by arrows), which confirm that IFN-γ inhibits M-CSF and RANKL-induced osteoclastogenesis.

FIG. 12 shows results of immunofluorescence staining for HIF1α and c-MET in metastatic bone tumors. Green represents HIF1α and red represents c-MET (scale bar=100 μm).

FIG. 13 shows results of Western blot performed using the same amount of protein after treating M6 and M6-bone cells with 100 ng/ml of IFN-γ for 18 hours, which may show that IFN-γ inhibits SOCS1 protein induction and oncogenic signaling.

FIG. 14 shows results of immunofluorescence staining for αSMA and CD31, which are angiogenesis-related markers in metastatic bone tumors. Green represents αSMA and red represents CD31 (scale bar: 20 μm). The expression of αSMA and CD31 in normal and metastatic bone tumors is expressed as mean±SEM (*, p<0.05).

FIG. 15 shows results of confirming a tumor growth inhibition effect through combined administration of a HIF1α/c-MET polypeptide vaccine and an immune checkpoint inhibitor. The mean tumor volume of a CFA/IFA group, a HIF1α/c-MET polypeptide vaccine alone group, a HIF1a/c-MET polypeptide+anti-PD-1 Ab group, and a HIF1α/c-MET polypeptide+anti-CTLA-4 Ab group was expressed as mean±SEM (**, p<0.005; ***, p<0.001).

FIG. 16 shows results of confirming the induction of IFN-γ secreting Th1 cells according to combined administration of a HIF1α/c-MET polypeptide vaccine and an immune checkpoint inhibitor. Each experimental group includes unstimulated cells and cells stimulated with a negative control peptide TT or HIF1α and c-MET peptides (***p<0.001).

FIG. 17 shows immunofluorescence staining images of CD4 and CD8 infiltrated into transplanted tumors in each group. CD4 and CD8 levels in normal and metastatic bone tumors are expressed as mean±SEM (*, p<0.05; ***, p<0.005).

FIG. 18 shows results of evaluating bone metastasis according to administration of a HIF1α/c-MET polypeptide vaccine and an immune checkpoint inhibitor in an advanced cancer metastasis model. FIG. 18A shows representative images confirming tumor growth and spread using luciferase imaging, and FIG. 18B shows quantification of the mean photon count in the bone region of mice in each group over time.

FIG. 19 shows results of evaluating the bone loss degree according to administration of a HIF1α/c-MET polypeptide vaccine and an immune checkpoint inhibitor in an advanced cancer metastasis model. Representative micro-CT of mouse bones in the CFA/IFA group, HIF1α/c-MET peptide alone group, HIF1α/c-MET peptide+anti-PD-1 Ab combination group, and HIF1α/c-MET peptide+anti-CTLA-4 Ab combination group are indicated by arrows.

FIG. 20 shows immunofluorescence staining images of CD4 and CD8 in each group to evaluate the degree of immune cell infiltration into metastatic bone tumors after administration of a HIF1α/c-MET polypeptide vaccine and an immune checkpoint inhibitor in an advanced cancer metastasis model. The expression of CD4 and CD8 in normal and metastatic bone tumors is expressed as mean±SEM (*, p<0.005; ***, p<0.001).

FIG. 21 shows results of measuring an Ag-specific T cell response in mouse splenocytes of each group by performing IFN-γ ELISPOT (***, P<0.001). The combined administration of the HIF1α/c-MET polypeptide vaccine and the immune checkpoint inhibitor induced IFN-γ-secreting Th1 cells.

FIG. 22 shows H&E and TRAP staining images of mouse bones in the CFA/IFA group, HIF1α/c-MET peptide alone group, HIF1α/c-MET peptide+anti-PD-1 Ab combination group, and HIF1α/c-MET peptide+anti-CTLA-4 Ab combination group (indicated by arrows). The combined administration of the HIF1α/c-MET polypeptide vaccine and the immune checkpoint inhibitor reduces TRAP-positive osteoclastogenesis.

FIG. 23 shows a cancer vaccine effect of a HIF1α/c-MET polypeptide in a lapatinib-resistant cancer animal model. Specifically, FIG. 23A is a schematic diagram of the experiment, and FIG. 23B is a graph of comparing the tumor sizes of experimental animals over time.

FIG. 24A shows results of confirming the induction of IFN-γ secreting Th1 cells by administration of a HIF1α/c-MET polypeptide in a lapatinib-resistant cancer animal model, and FIGS. 24B to 24D show H&E and immunofluorescence staining images of the tumor tissues of the animals.

FIG. 25 is a schematic diagram of an experiment using a systemic metastatic cancer-induced animal model by administration of a M6-bone cell line.

FIGS. 26A and 26B show results of H&E and immunofluorescence staining of lung, liver, and bone tissues of a systemic metastatic cancer-induced animal model, which confirm the occurrence of metastatic cancer in the lung, liver, and bone by administration of the M6-bone cell line.

FIG. 27 shows luciferase imaging images of evaluating cancer metastasis according to administration of a HIF1α/c-MET polypeptide vaccine and an immune checkpoint inhibitor in a systemic metastatic cancer-induced animal model.

FIG. 28A shows H&E and immunofluorescence staining images confirming CD47 overexpression in lung and liver tissues of a systemic metastatic cancer-induced animal model, and FIG. 28B shows results of confirming CD47 overexpression in the bones of the animal model and graphs of quantifying the number of CD47 overexpressing cells in the lung, liver, and bone.

FIGS. 29A and 29B show H&E and immunofluorescence staining images and quantitative graphs confirming CD8 T cells infiltrated into tumor tissues according to administration of a HIF1α/c-MET polypeptide vaccine and an immune checkpoint inhibitor in a systemic metastatic cancer-induced animal model.

### BEST MODE FOR CARRYING OUT THE INVENTION

Patients with bone metastasis TNBC who do not respond to targeted therapy have a higher mortality rate compared with patients with HR or HER2 breast cancer. It has been reported that c-MET and HIF-1α proteins were overexpressed in 52% and 80% of TNBC patients, respectively, to have a potential as immunological targets. The present inventors constructed a bone metastasis model using a TNBC mouse model and investigated a bone microenvironment. As a result, it was confirmed that targeted immunization using the HIF1α/c-MET polypeptide vaccine significantly inhibited metastatic bone tumors by inducing antigen-specific T cells in the bone microenvironment and increasing the infiltration into the tumors. Subsequently, a decrease in the expression of target proteins HIF1α and c-MET and a decrease in the expression of angiogenic markers in tumors were confirmed immunized with the vaccine. Furthermore, an effective immune response was confirmed in mice exhibiting reduced osteolysis by reducing the formation of osteoclasts in the bone microenvironment. Finally, as a result of combining the HIF1α/c-MET vaccine and an immune checkpoint inhibitor, a potential as a therapeutic vaccine was confirmed in an advanced cancer environment. From the results, the present inventors intend to provide a HIF1α/c-MET polypeptide as a cancer vaccine and a method for treating cancer through co-administration of the cancer vaccine and an immune checkpoint inhibitor.

Conventional bone metastasis cancer studies have been conducted by implanting human breast cancer cell lines into immunodeficiency mice. Research models using immunodeficiency mice have limitations of overlooking the interaction between the bone microenvironment and immune cell responses. To solve these problems, the present inventors established a bone metastasis model using a bone metastatic cell line (hereinafter referred to as an M-bone) in C3(1)-Tag mice as a TNBC model, and demonstrated that the interaction between cancer cells and the bone microenvironment plays an important role in the bone metastasis process. Particularly, it was confirmed that bone metastasis was associated with a decrease in T cells and an increase in MDSCs. The bone metastasis may evade immune surveillance in the bone microenvironment by suppressing type I IFN signals and inducing MDSCs.

An MHC class I-restricted vaccine does not always induce immune responses sufficient to induce effective antitumor immunity. The MHC class I-based vaccine may induce tolerance or anergy of CD8 T cells when presented by MHC class I molecules expressed on non-professional APCs due to the lack of co-stimulatory molecule signaling. This phenomenon limits the effect of short peptide vaccines. One of the reported weaknesses of TAA-derived MHC class I-based vaccines is that the clinical efficacy is limited by inducing not only Type I T cells but also Type II T cells or Treg cells. In contrast, the MHC class II epitope-based vaccine may not directly bind to MHC class I molecules expressed on non-professional APCs to overcome these problems.

Immunological regulation of TNBC requires robust immune responses to various antigens. In addition, it has been reported that multi-epitope vaccines induce stronger Th1-type immune responses compared to single-epitope vaccines to significantly reduce the tumor volume, limit bone destruction, and significantly improve survival rates. Multi-epitope and multi-peptide-based vaccines may be more suitable therapeutic agents than single-epitope and single-peptide-based vaccines by inducing stronger immunity and avoiding immune escape. For this reason, the present inventors selected epitopes targeting c-MET and HIF1α to be combined into a polypeptide vaccine. The epitope targeting HIF1α was used as developed in previous studies, and the epitope targeting c-MET was used with an epitope verified by selecting a peptide sequence with high affinity for HLA in the c-MET protein, performing INF-gamma ELISPOT and IL-10 ELISPOT to induce a Th1 immune response, and induce the immune response in mice. The amino acid sequence of the used c-MET epitope is as follows.

-p275-289:
HTRIIRFCSINSGLH
-p314-328:
FNILQAAYVSKPGAQ

Accordingly, the present inventors provide a polypeptide in which epitopes of c-MET and HIF1α are fused as a cancer vaccine for preventing and/or treating cancer.

TABLE 1

| | Amino acid sequence | SEQ ID NO: |
|---|---|---|
| cMet epitope | HTRIIRFCSINSGLH (P275) | 1 |
| | FNILQAAYVSKPGAQ (P314) | 2 |
| HIF1 alpha epitope | YELAHOLPLPHNVSSH (p38-53) | 3 |
| | MRLTISYLRVRKLLDAGDLDIED (p60-82) | 4 |
| | LKALDGFVMVLTDDGDMIYISDNVN (p93-117) | 5 |
| HIF1α/cMet | HTRIIRFCSINSGLH (P275) | 6 |
| polypeptide | FNILQAAYVSKPGAQ (P314) | |
| | YELAHQLPLPHNVSSH (p38-53) | |
| | MRLTISYLRVRKLLDAGDLDIED (p60-82) | |
| | LKALDGFVMVLTDDGDMIYISDNVN (p93-117) | |

Tumor cells evade immune surveillance and suppress antitumor immune responses through various mechanisms, including activation of immune checkpoint pathways. The immune checkpoint inhibitor hinders co-inhibitory signaling pathways to activate antitumor immune responses and promote immune-mediated clearance of tumor cells. The immune checkpoint inhibitor is a breakthrough in cancer treatment, but the clinical effect is still limited to some patients. The blocking of the immune checkpoint pathways is most effective when there is a conventional tumor-specific T cell response, but the low immunogenicity of many tumors does not sufficiently induce CD8 T cell-mediated antitumor immunity. Vaccination may induce expansion of tumor-specific T cells and enhance antitumor immune responses. Herein, the present inventors studied the antitumor effect and bone metastasis inhibition of Type I immunity using the vaccine of the present disclosure, which was confirmed to induce Th1, and simultaneously evaluated the effect of combined treatment with the immune checkpoint inhibitor in C3(I)-Tag mice. As a result, it was confirmed that when administering a vaccine in combination with an immune checkpoint inhibitor, more tumor trafficking of Th1 is induced, resulting in effective antitumor response and inhibition of bone metastasis.

Meanwhile, an interferon gene is an indicator that may predict a better prognosis in various carcinomas, including breast cancer, pancreatic cancer, and ovarian cancer. In particular, increased IFN-γ in cancer is associated with positive response and outcome to treatment. In tumors, IFN-γ is known to inhibit cell proliferation and angiogenesis and induce apoptosis. In addition, local IFN-γ upregulation has been known to be essential for anti-PD-1-mediated tumor suppression in mouse models. In addition, IFN-γ inhibits osteoclastogenesis by inhibiting RNAK signaling in osteoclast precursors of mice. In contrast, loss of the IFN-γ receptor induces osteoclastogenesis and enhances bone destruction in an inflammatory bone loss mouse model. That is, IFN-γ may prevent tumor-associated bone loss by inhibiting osteoclastogenesis. The present inventors confirmed that the number of osteoclasts was significantly reduced in immunized mice with bone metastatic tumors compared to a control group. In vitro experiments on osteoclastogenesis using bone marrow monocytes (BMMs) expressing C3(1)-Tag supported the role of IFN-γ. These data suggest that an effective immune response secreting IFN-γ may regulate osteoclastogenesis in response to metastatic cancer in the bone microenvironment in vivo.

Meanwhile, the present inventors confirmed that the developed cancer vaccine induced a decrease in the expression of oncoproteins, c-MET and HIF1α, and a decrease in the expression of angiogenesis-related markers.

Hypoxia inducible factor (HIF)-1 is a transcription factor that maintains intracellular homeostasis by inducing glycolysis and angiogenesis processes to appropriately respond to changes in external oxygen concentration in hypoxic conditions, and overexpression is known in various solid cancers, including colon cancer, liver cancer, gastric cancer, and breast cancer, and small molecule substances targeting HIF-1alpha as a subtype of HIF-1 are in clinical trials. In addition, it has been known that in the bone metastasis process, HIF-1α signaling contributes to bone metastasis by inhibiting differentiation of osteoblasts and promoting osteoclastogenesis to regulate the bone microenvironment.

C-Met is known to be involved in the carcinogenesis by being activated by a hepatocyte growth factor (HGF) to continuously activate the intracellular signaling system within the cell. The expression of c-Met is known to be increased in cancer types such as liver cancer, lung cancer, stomach cancer, thyroid cancer, prostate cancer, endometrial cancer, and breast cancer.

In the present disclosure, the “epitope” is a set of amino acid residues in an antigen binding site recognized by a specific antibody, or residues recognized by a T cell receptor protein and/or a Major Histocompatibility Complex (MHC) receptor in a T cell. The epitope is a molecule that forms a site recognized by an antibody, a T cell receptor, or an HLA molecule, and refers to a primary, secondary, and tertiary peptide structure, or charge.

The present disclosure provides a polypeptide including a HIF-1alpha epitope and a c-Met epitope as a cancer vaccine. In the present disclosure, the cancer vaccine may be provided as a polypeptide in which the peptides of the two epitopes are linked, and the peptides of the two epitopes may exist separately, but may be provided as a single composition, and the peptides of the two epitopes may also be provided separately as separate compositions to be used in combination.

In the present disclosure, the epitope of HIF-1α and the epitope peptide of c-MET constituting the cancer vaccine may be prepared by known chemical synthesis methods or genetic engineering methods, and may include modifications at an amino (N—) terminus or carboxyl (C—) terminus to improve stability. The “stability” refers to not only in vivo stability, but also storage stability (including storage stability at room temperature, refrigeration, and frozen storage).

In this specification, the polypeptide including the HIF-1α epitope and the c-Met epitope is referred to as a “HIF1α/c-Met polypeptide”, and is not limited as long as the polypeptide is injected into the body and activates an immune response by providing the HIF1α epitope and the c-Met epitope, and includes two types of peptides that exist separately from one polypeptide in which each epitope is linked as described above. The cancer vaccine of the present disclosure may include a genetic material encoding the polypeptide or peptide as well as the polypeptide and peptide itself.

In the present disclosure, when the genetic material encoding the polypeptide or peptide is provided as the cancer vaccine, the genetic material may consist of RNA and/or DNA and may include a modified nucleotide. When the genetic material mainly consists of RNA, the genetic material may include known components for expression in vivo, and non-limiting examples thereof include IRES, 5′-Capping, and the like. In addition, when the genetic material mainly consists of DNA, the genetic material may be provided as a recombinant vector including an operably linked promoter for its expression. In addition, the genetic material may include a signal peptide for secretion outside the cell.

As used in the present disclosure, the “promoter” is involved in the binding of RNA polymerase to initiate transcription as a portion of DNA. Generally, the promoter is a site that is adjacent to the target gene and located upstream of the target gene, and bound with RNA polymerase or a transcription factor which is a protein inducing RNA polymerase and may induce the enzyme or protein to be located at a correct transcription starting site. That is, the promoter has a specific gene sequence that is located at a 5′ site of a gene to be transcribed in a sense strand so that RNA polymerase binds to the corresponding position directly or through a transcription factor to initiate mRNA synthesis for the target gene.

As used in the present disclosure, the “cancer” to be prevented or treated by the vaccine means a condition in which there is a problem in a regulatory function of normal division, differentiation, and apoptosis of cells, and thus the cells are abnormally excessively proliferated and infiltrated to surrounding tissues and organs to form a lump and destroy or transform an existing structure.

In the present disclosure, the cancer may include solid cancers in which overexpression of c-MET and/or HIF-1α has been reported, such as non-small cell lung cancer, stomach cancer, head and neck cancer, kidney cancer, liver cancer, prostate cancer, and thyroid cancer, in addition to breast cancer, and further, the vaccine of the present disclosure may prevent or treat metastatic cancer, particularly bone metastatic cancer.

The cancer vaccine of the present disclosure induces a Th1 immune response.

As used in the present disclosure, the “Th1 cell” refers to a subset of helper T cell lymphocytes that are characterized in terms of gene expression, protein secretion, and functional activity. For example, the Th1 cell exhibits a cytokine expression pattern that synthesizes IL-2 and IFN-γ, but does not synthesize IL-4, IL-5, IL-10, and IL-13. The Th1 cells are involved in cell-mediated immune responses to various intracellular pathogens, organ-specific autoimmune diseases, and delayed hypersensitivity reactions.

In addition, the cancer vaccine of the present disclosure may exhibit a more remarkable anticancer effect when administered in combination with the immune checkpoint inhibitor, and improve drug sensitivity in immune checkpoint inhibitor-resistant cancer.

Accordingly, the present disclosure provides a combination therapy of the above-described cancer vaccine and the immune checkpoint inhibitor for the prevention or treatment of cancer. At this time, the cancer vaccine and the immune checkpoint inhibitor may be provided as a single composition, or may be provided separately and administered sequentially. When the cancer vaccine and the immune checkpoint inhibitor are administered sequentially, the order is irrelevant, but desirably, the immune checkpoint inhibitor may be administered after the cancer vaccine is administered.

In the present disclosure, the immune checkpoint inhibitor (ICI) performs an inhibition function of binding of PD-L1 and PD-1 to maintain the immune function of T cells and inhibits PD-1 or PDL-1, which inhibits the activity of T-cells infiltrated into tumors to maximize the activity of T-cells, thereby increasing the anticancer effect. In the present disclosure, it was confirmed in a specific experiment that the vaccine of the present disclosure and anti-CTLA-4 Ab or anti-PD-1 Ab were administered in combination to have effects of increasing immune cell infiltration into tumors and inhibiting tumor growth. However, the immune checkpoint inhibitor is not limited as long as the immune checkpoint inhibitor is an immune checkpoint inhibitor that targets immune checkpoint proteins for T-cell activation. In the present disclosure, the immune checkpoint inhibitor may be an inhibitor of CTLA-4, PD-1, PD-L1, PD-L2, LAG-3, BTLA, B7H3, B7H4, TIM3, KIR, TIGIT, CD47, VISTA or A2aR.

As used in the present disclosure, the “prevention” means all actions that inhibit metastasis of cancer or delay the onset of cancer and its metastasis by administering a vaccine according to the present disclosure or a combination of the vaccine and an immune checkpoint inhibitor.

As used in the present disclosure, the “treatment” means all actions that alleviate cancer or beneficially change its symptoms by administering a vaccine according to the present disclosure or a combination of the vaccine and an immune checkpoint inhibitor.

The vaccine of the present disclosure may be provided as a pharmaceutical composition including the above-described HIF1α/c-Met polypeptide or each peptide, and the pharmaceutical composition according to the present disclosure may further include a pharmaceutically acceptable carrier, excipient or diluent. Examples of the pharmaceutically acceptable carrier, the excipient, and the diluent that may be used in the pharmaceutical composition of the present disclosure may include lactose, dextrose, sucrose, sorbitol, mannitol, xylitol, erythritol, maltitol, starch, acacia gum, alginate, gelatin, calcium phosphate, calcium silicate, calcium carbonate, cellulose, methylcellulose, polyvinylpyrrolidone, water, methylhydroxybenzoate, propylhydroxybenzoate, talc, magnesium stearate, mineral oils, and the like.

The pharmaceutical composition of the present disclosure may be administered orally or parenterally depending on a desired method, but is desirably administered parenterally.

According to an example of the present disclosure, the pharmaceutical composition according to the present disclosure may be directly administered intravenously, intraarterially, subcutaneously, or into cancer tissue, or administered as injections. The injection according to the present disclosure may be a form dispersed in a sterile medium so as to be used as it is when administered to a patient, or may also be administered after dispersing in an appropriate concentration by adding distilled water for injection during administration. In addition, when prepared as the injection, the pharmaceutical composition may be mixed with buffers, preservatives, analgesics, solubilizers, isotonicity agents, stabilizers, etc., and may be prepared in unit dosage ampoules or multiple dosage forms.

A dose of the pharmaceutical composition of the present disclosure varies according to the condition and body weight of a patient, the degree of a disease, a drug form, and the route and period of administration, but may be properly selected by those skilled in the art. Meanwhile, the pharmaceutical composition according to the present disclosure may be used alone or in combination with auxiliary therapy methods such as surgical therapy.

In the present disclosure, an amino acid sequence is abbreviated as follows according to the IUPAC-IUB nomenclature.

Arginine (Arg, R), Lysine (Lys, K), histidine (His, H), serine (Ser, S), threonine (Thr, T), glutamine (Gln, Q), asparagine (Asp, N), methionine (Met, M), leucine (Leu, L), isoleucine (Ile, I), valine (Val, V), phenylalanine (Phe, F), tryptophan (Trp, W), tyrosine (Tyr, Y), alanine (Ala, A), glycine (Gly, G), proline (Pro, P), cysteine (Cys, C), aspartic acid (Asp, D), glutamic acid (Glu, E), norleucine (Nle)

### MODE FOR CARRYING OUT THE INVENTION

Hereinafter, examples will be described in detail with reference to the accompanying drawings. However, since various modifications may be made to examples, the scope of the present disclosure is not limited or restricted by these examples. It should be understood that all modifications, equivalents and substitutes for examples are included in the scope of the present disclosure.

The terms used in examples are used for the purpose of description only, and should not be construed to be limited. The singular expression includes the plural expression unless the context clearly dictates otherwise. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which examples pertain. Terms which are defined in a generally used dictionary should be interpreted to have the same meaning as the meaning in the context of the related art, and are not interpreted as ideal or excessively formal meanings unless otherwise defined in the present disclosure.

In addition, in the description with reference to the accompanying drawings, like components designate like reference numerals regardless of reference numerals and a duplicated description thereof will be omitted. In describing the examples, a detailed description of related known technologies will be omitted if it is determined that they unnecessarily make the gist of the examples unclear.

### Experimental Methods and Materials

### 1. Animal Model

5 to 8 week-old female FVB-Tg (C3-1-TAg) cJeg/Jeg (C3(1)-Tag) mice were purchased from Jackson Laboratories. C3(1)-Tag mice were a TNBC mouse model with a basic phenotype. Each experimental group used at least four mice. All experiments followed protocols approved by the Korea University Animal Laboratory, and all mice were raised in a sterile facility. (Korea-2020-0014)

### 2. Isolation of Bone Metastatic Cells and Establishment of Cell Lines

A mouse mammary gland tumor cell line M6 was derived from a spontaneous mammary gland tumor of C3(1)-Tag mice. M6-luc cells were inoculated into C3(1)-Tag mice by intracardiac injection. After two weeks, tibial metastatic lesions were identified by in vitro bioluminescence imaging and the metastatic sites were resected from the mice. After the entire hind limb was cut with a blade, the cut hind limb was cultured in an RPMI1640 medium for 24 hours. The next day, cells attached to a dish were identified and treated with 10 μg/ml blasticidin in an RPMI1460 medium containing 10% FBS and 1% antibiotics to select only metastatic cells. All cell lines were cultured in the RPMI 1640 medium supplemented with 10% FBS and 1% antibiotics and maintained at 37° C. in a 5% CO₂ incubator. This process was repeated twice to establish a bone seeking cell line.

3. Construction of Triple-Negative Breast Cancer Mouse Model with Bone Metastasis

A bone metastatic cell line was cultured in an RPMI1460 medium supplemented with 10% FBS and 10 μg/ml blasticidin and stably transfected with luciferase (LVP326, GenTaget). For bone metastasis formation, 1×10⁵ viable cells were washed, harvested in DPBS, and inoculated into 8 to 9 week-old C3(1)-Tag mice by intracardiac injection. Bone metastasis was monitored every week by bioluminescence imaging using the NightOWL LB 983 in vivo imaging system. Four weeks after inoculation, C3(1)-Tag mice were harvested at week 6.

### 4. RNA Sequencing

Total RNA samples were transformed into a cDNA library using the TruSeq Stranded mRNA Sample Prep Kit (Illumina). Starting with 1000 ng of total RNA, mRNA was mainly selected and purified using oligo-dT-conjugated magnetic beads. The purified mRNA was physically fragmented and single-stranded cDNA was constructed using reverse transcriptase and a random hexamer primer. Actinomycin D was added to inhibit DNA-dependent synthesis of the second strand, and double-stranded cDNA was constructed by synthesizing the second strand in the presence of deoxyribouridine triphosphate (dUTP) instead of deoxythymidine triphosphate (dTTP) after removing the RNA template. A single A base was added to the 3′ end to facilitate ligation of a sequencing adapter containing a single T base overhang. Adapter-ligated cDNA was amplified by polymerase chain reaction to increase the amount of sequence-ready library. During PCR, the polymerase stopped when encountering the U base, making the second strand a bad template. Therefore, the amplified material preserved strand information by using the first strand as a template. The final cDNA library was analyzed for size distribution using an Agilent Bioanalyzer (DNA 1000 Kit; Agilent), quantified by qPCR (Kapa Library Quant Kit; Kapa Biosystems, Wilmington, MA), and normalized to 2 nmol/L for sequencing. The indexed library was sequenced using the Novaseq 6000 platform (Illumina, San Diego, USA, Macrogen Inc).

### 5. Induction of Immune Responses and Tumor Growth

Each mouse was vaccinated with complete/incomplete Freud's adjuvant (sigma), 50 μl of HIF-1α peptide pools (p38-53, p60-82, and p93-117; 50 μg) and c-MET peptide pools (p275, p314; 50 μg each). To induce an effective immune response against an overexpressed autoantigen, three vaccinations were performed at 7 to 10 day intervals. Then, corresponding syngenic M6 cells (0.5×10⁶ cells) for tumor attack were implanted into a breast fat pad 10 days after the last vaccination (n=5/group). Tumors were measured in the same manner as described above. All tumor growth was expressed as mean tumor volume (mm³±SEM).

### 6. In Vivo PD-1/CTLA-4 Blockade Therapy

M6 or M6-bone tumor cells were injected into mice before starting PD-1/CTLA-4 blockade therapy. Then, mice were injected intraperitoneally once every 3 days with Armenian hamster IgG (BioXCell)/rat IgG2b (LTF-2, BioXCell) isotype control antibodies, anti-PD-1 blocking antibody (250 μg/mouse, J43, BioXCell), and anti-CTLA-4 blocking antibody (150 μg/mouse, UC10-4F10-11, BioXCell).

### 7. H&E Staining

After the experiment was completed, the mice were sacrificed, and the lung, liver, bone, and tumors were isolated and fixed in 4% formalin. H&E staining was performed in the pathology department as follows: After dehydration, the tissue sections were soaked in hematoxylin for 10 minutes, washed with tap water for 1 minute, and then quickly immersed in HCl alcohol, washed with tap water for 1 minute, immersed in ammonia water for 1 minute, immersed in eosin for 15 seconds, and then dehydrated. The tissue sections were immersed and dehydrated in a series of increasing concentrations of alcohol (95% twice and 100% twice, each for 1 minute). Thereafter, each section was immersed twice in xylene for 1 minute each, mounted with Permount, and photographed using an optical microscope.

### 8. Immunofluorescence, Immunohistochemical, and TRAP Stainings

Tissues fixed in paraffin were cut onto slides, and paraffin was removed from the slides using xylene and ethanol. Slide tissues were incubated overnight at 4° C. with primary antibodies: Rabbit anti-CD4, mouse anti-CD8, anti-HIF1α, (NB100-105, Novusbio), anti-c-MET (ab51067, Abcam), anti-CD31, anti-αSMA (C6198, Sigma) diluted in background block serum. The slide tissues with anti-CD4, anti-CD8, anti-HIF1α, and anti-c-MET primary antibodies were washed three times with TBS and incubated with the following secondary antibody for 1 hour: Alexa Fluor®488 rat anti-mouse IgG (1:1000). Nuclei were stained with 1 μg/ml DAPI (1:1000, D1306, Invitrogen). The stained tissue slides were washed three times with TBS and mounted with a fluorescent mounting medium (S3023, Dako).

Tissue slides with anti-HIF1α and anti-c-MET were incubated with a nonfluorescent second antibody for 1 hour at room temperature. A DAB solution was incubated for 3 minutes, washed with D.W, and nuclei were stained with hematoxylin. The stained tissue slides were washed with D.W, dehydrated, and then mounted. Osteoclast staining was performed using a TRAP staining kit (KT-008, KAMIYA biomedical company, Seattle, WA, USA).

### 9. Enzyme-Linked Immune Spot Assay (ELISPOT)

After the mice were sacrificed, splenocytes were isolated from the spleen. ELISPOT analysis was performed to evaluate the frequency of IFN-γ secreted from Ag-specific T cells. After reacting with 30 μl/well of 35% ethanol for 1 minute in a 96-well filtration plate (MAIPS4510, Merck Millipore, Darmstadt, Germany), 200 μl was washed three times with 1×PBS. Next, 10 μg/ml anti-mouse IFN-γ antibody (AN81, MabTech, Stockholm) was coated on a 96-well filtration plate at 50 μl per well overnight at 4° C. The cells were cultured overnight at 4° C. and then washed three times with 1×PBS and incubated with 200 μl of a mouse T cell medium at room temperature for 2 hours.

The mouse T cell medium was removed, and then 2.5 or 3×10⁶ splenocytes were plated. The splenocytes were cultured in a medium containing 10 μg/ml TT peptide, 10 μg/ml HIF1α peptide, 10 μg/ml c-MET peptide, and 5 μg/ml concanavalin A (Sigma-Aldrich) in an incubator at 37° C. for 48 to 72 hours. Thereafter, the plate was washed with 200 μl of PBS dissolved in 0.05% Tween-20, and added with 5 μg/ml of biotinylated anti-mouse IFN-γ antibody (R46A2, MabTech) at 50 μl per well and cultured overnight at 4° C. The plate was scanned and spots were counted using an automated ELISPOT reader system.

### 10. Osteolastogenesis Analysis

Primary mouse bone marrow monocytes (BMMs) were isolated from the hind limb bones (two femurs and two tibiae) of 4 to 8 week-old C3(1)-Tag mice. The mouse BMMs were flushed into a 15 ml tube via 8 ml of a serum-free 2 mM EDTA α-MEM medium. A Lymphocyte Separation Medium (LSM; MP Biomedicals; Catalog No. 50494) was dispensed into a new tube, 8 ml of the flushed BMMs were gently added thereon and centrifuged at 1600 rpm for 20 minutes at room temperature. The isolated BMMs were inoculated at 3×10⁵ cells/well in a 48-well plate with 0.5 mL of a medium per well and incubated in α-MEM containing 10% FBS and 1% antibiotics.

BMMs in a negative control group were treated with M-CSF (30 ng/mL), BMMs in a positive control group were treated with M-CSF and RANKL (100 ng/mL), and BMMs in an experimental group were stimulated by treatment with M-CSF, RANKL, and IFN-γ(50, 100 ng/mL). On days 1, 3, 5, and 7 of culture, the medium was replaced, and each experimental group was treated with M-CSF, RANKL, and IFN-γ. After 9 days of culture, staining was performed using a TRAP staining kit (KT-008, KAMIYA biomedical company, Seattle, WA, USA) after fixing the cells with 10% formalin. For each well, TRAP-positive (TRAP⁺) multinucleated cells (TRAP⁺ MNC) were counted. All the experiments were independently repeated three times.

### 11. Western Blotting

M6 and M6-bone cells were stimulated with 100 ng/ml recombinant mouse IFN-γ and cultured in a medium containing 1% FBS for 18 hours. The cells were lysed in a buffered mixture of a PRO-PREPTM protein extraction solution (iNtRON Biotechnology, 17081) and a phosphatase inhibitor cocktail (Gen DEPOT, p3200-001). Protein concentration was measured using the Bradford assay (Bio-rad, 500-0006, Hercules, CA, USA). 30 μg of the protein was separated by SDS-polyacrylamide gel electrophoresis and transferred to a PVDF membrane (10600023), and then the membrane was blocked with Tris-buffered saline containing 0.05% Tween 20 and 5% non-fat milk powder for 1 hour. The PVDF membrane was incubated overnight at 4° C. with primary antibodies. HIF1α (NB100-105, Novusbio), c-MET, Akt (Cat #9272), and p-Akt (Cat #9271) antibodies were purchased from Cell Signaling Technology (Beverly, MA, USA), and β-actin (Cat #A5316) was purchased from Sigma (Saint Louis, MO, USA). Subsequently, a horseradish peroxidase-conjugated secondary antibody was cultured at room temperature for 1 hour. Immunoreactive bands were detected with a chemiluminescent reagent (Cat #RPN2106).

### 12. Statistical Analysis

Graphs and statistical comparison were performed using Graph-pad Prism v5.01 software. Differences between a plurality of experimental groups were analyzed using one-way or two-way ANOVA followed by a turkey test.

### Experiment Results

### 1. Construction of Bone Metastasis Mouse Model

To investigate the mechanism and microenvironment of bone metastasis in breast cancer, bone metastatic cells were constructed using M6 cells generated from spontaneous breast tumors in a C3(1)-Tag mouse model. Specifically, M6 cells were injected intracardiacly into 8-9 week old C3(1)-Tag female mice, and the cells were isolated and cultured from bone metastatic lesions to establish a bone seeking cell line, which was named M6-bone. The M6-bone cells were reinjected intracardiacly for another cycle of in vivo selection and culture. M6-bone cell migration was confirmed by tracking fluorescence 14 days after intracardiac administration (FIG. 1A). Subsequently, the hind limb of the metastatic mice were collected and measured by X-ray. X-rays showed dramatic destruction of the metastatic hind limb compared with the control hind limb (FIG. 1B). In addition, through histological analysis, the presence of cancer cells was confirmed in the liver, lung, and bone (FIG. 1C).

2. Confirmation of Relation of HIF1α and c-MET with Bone Metastasis

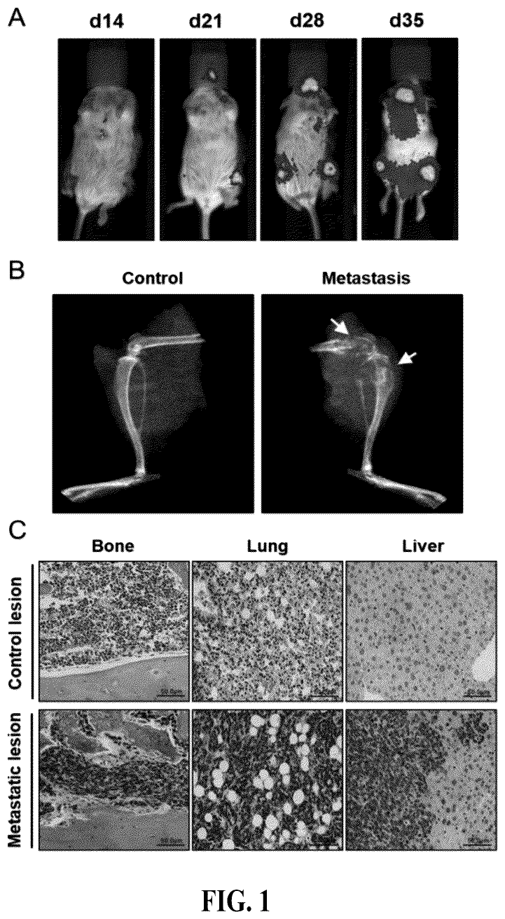

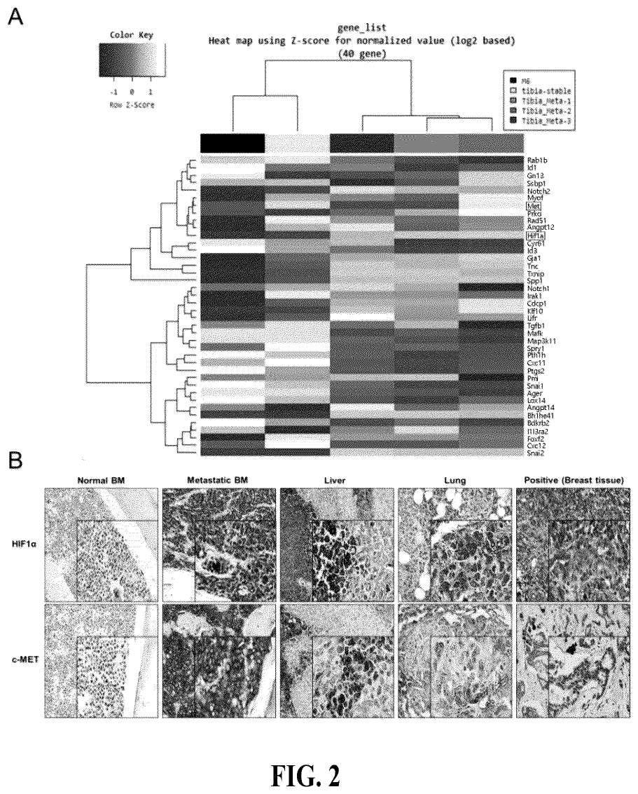

To determine a mechanism of bone metastasis, RNA sequencing was performed using metastatic bone, M6 cells, and M6-bone cells. Expression of HIF1α and c-MET was increased in metastatic bone compared to M6-bone clone (FIG. 2A). Protein levels of two biological markers, HIF1α and c-MET, in metastatic bone tumors were compared with those in normal bone, liver, and lung using immunohistochemical staining (FIG. 2B).

### 3. Evaluation of Metastatic Bone Cancer Microenvironment

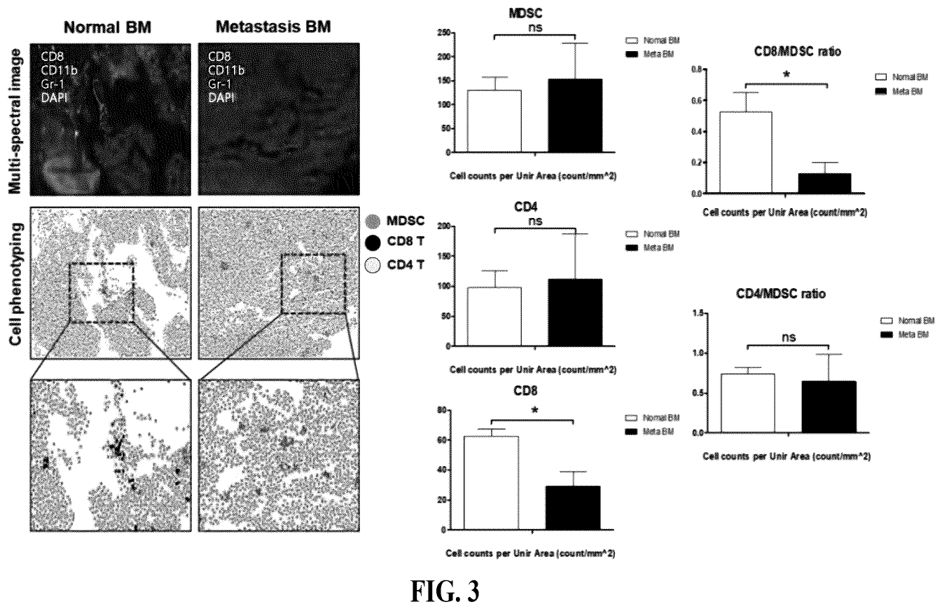

Immune cell infiltration in metastatic bone tumors was evaluated by multiple immunohistochemistry. Infiltration of CD4 T cells (98.2 vs 111.7 counts/HPF), CD8 T cells (62.8 vs 29.5 counts/HPF), and myeloid-derived suppressor cells (MDSCs) (Gr-1+/CD11b+) (130 vs 153.7 counts/HPF) was observed in normal and metastatic bone marrows. As shown in FIG. 3, after cancer cells metastasized to the bone, fewer CD8 T cells but more MDSCs were infiltrated than normal bone marrow. In summary, a CD8/MDSC ratio (0.12 vs 0.52, p<0.05) was significantly lower in the metastatic bone marrow than in the normal bone marrow, while a CD4/MDSC ratio (0.74 vs 0.64, ns) did not show significant results in the metastatic bone marrow compared with the normal bone marrow.

4. Confirmation of Tumor Growth Inhibition of HIF1α and c-MET Polypeptide Vaccines in Early Breast Cancer Model

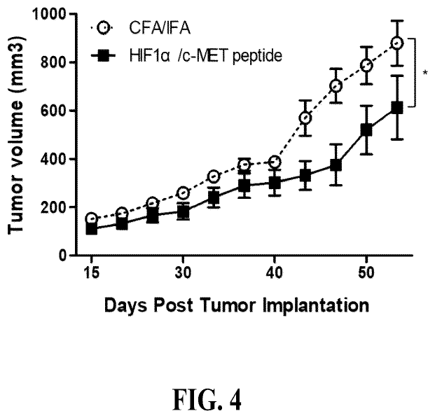

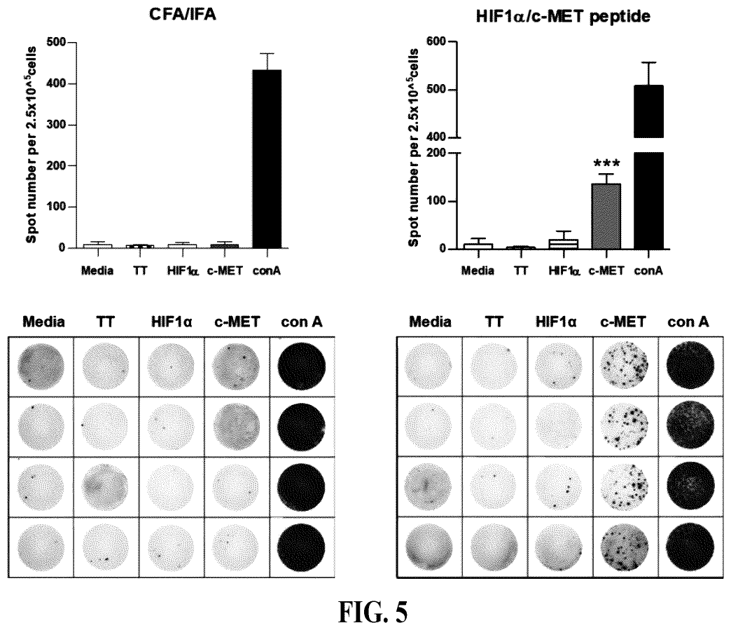

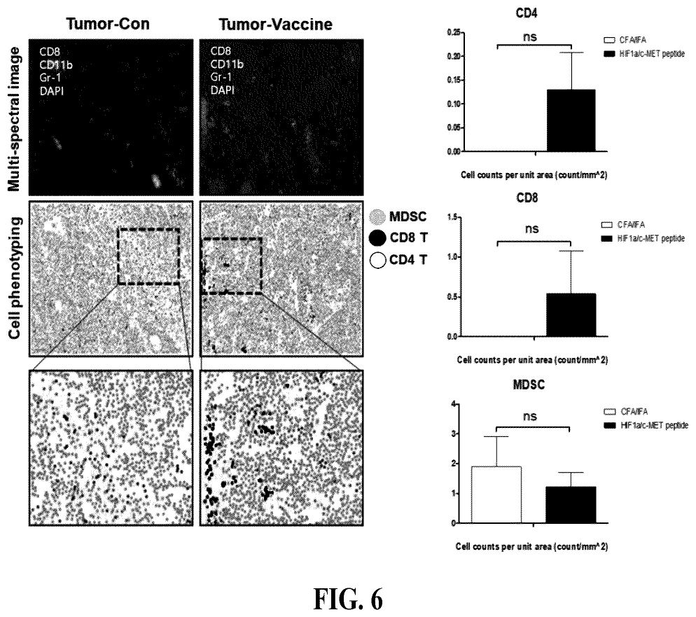

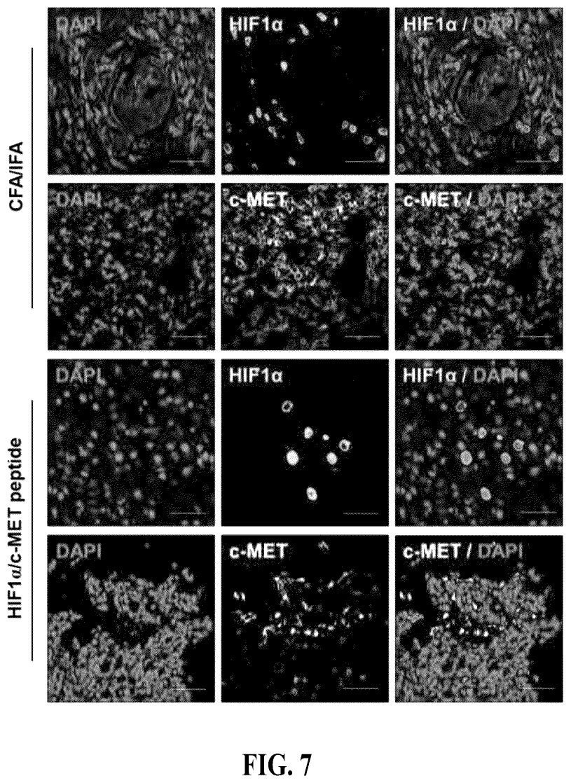

Whether the HIF1α/c-MET polypeptide vaccine affected tumor growth was examined. As a result of serially measuring M6 tumors implanted into immunized mice, it was confirmed that tumor growth was restricted in a HIF1α/c-MET polypeptide vaccine group compared to a control group (613±131 mm³ vs 879±93 mm³, p<0.05) (FIG. 4). The ability of the vaccine to induce interferon (IFN)-γ-secreting antigen-specific T cells was confirmed using ELISPOT. Compared with the control group, the HIF1α/c-MET polypeptide vaccine induced a significantly higher level of antigen-specific Type I T cells. In splenocytes, antigen-specific T cell responses were more prominent when stimulated with the c-MET peptide than with the HIF1α peptide (FIG. 5). To evaluate the tumor microenvironment, T cell infiltration in the implanted tumors was evaluated by immunohistochemistry. The MDSC infiltration (1.9 vs 1.22 counts/HPF, ns) was reduced, but CD8 T cell infiltration (0 vs 0.54 counts/HPF, ns) was increased in tumors of the vaccine group compared to the control group (FIG. 6). However, no statistical significance was found. Expression of two target proteins c-MET and HIF1α of the vaccine was significantly reduced in immunized tumors compared to a control tumor (FIG. 7). From the results, it could be seen that the HIF1α/c-MET polypeptide vaccine targeting c-MET and HIF1α restricted tumor growth and increased Ag-specific T cell immune responses, and furthermore, the immunized tumors showed increased CD8 T cell infiltration and decreased target protein expression.

5. Confirmation of Bone Metastasis Inhibition Effect of HIF1α and c-MET Polypeptide Vaccines in Early Breast Cancer Model

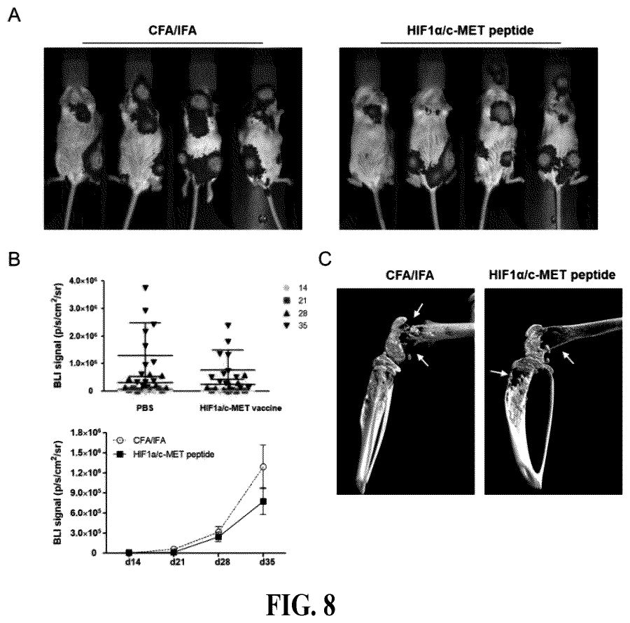

To examine the effect of the HIF1α/c-MET polypeptide vaccine, a systemic metastasis model was constructed by intracardiac injection of the M6-bone clone after three vaccinations. A luciferase signal detected in the tibia on day 35 after injection was lower in the vaccine group than in the control group (FIGS. 8A and 8B). The effect of the HIF1α/c-MET polypeptide vaccine on bone loss was evaluated by microcomputed tomography (CT) analysis. In three-dimensionally reconstructed micro-CT images of bones from a representative control animal, severe bone destruction was shown. In contrast, the bones in the vaccinated group showed limited osteolysis with intact periosteum integrity (FIG. 8).

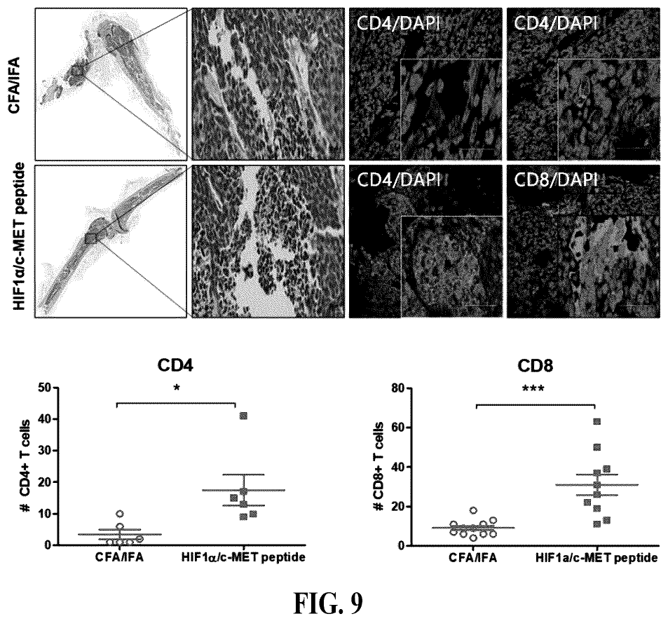

In addition, T cell infiltration was evaluated by immunofluorescence staining. The number of infiltrated CD4 (mean; 3.5 vs 23.6 counts, p<0.05) and CD8 T cells (mean; 9 vs 31.1 counts, p<0.001) was significantly increased in a HIF1α/c-MET polypeptide vaccine group compared to a CFA/IFA group (FIG. 9).

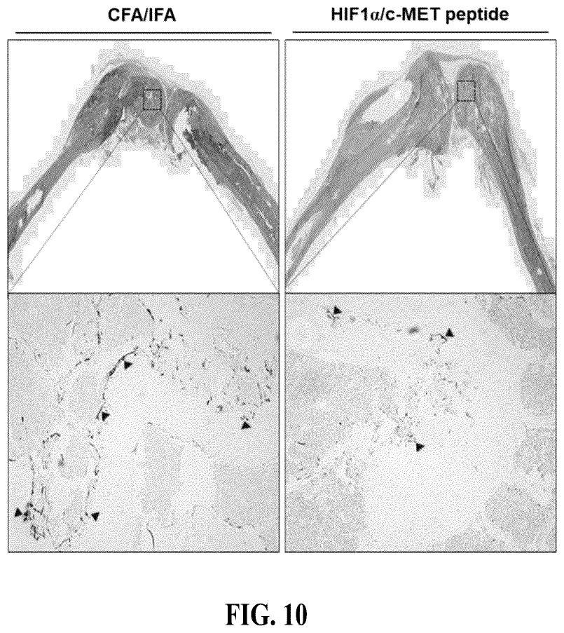

Because reduced osteolysis was observed in the vaccine group, osteoclasts were further evaluated in the bone using TRAP staining. The number and activity of osteoclasts in the HIF1α/c-MET polypeptide vaccine group were significantly reduced compared to the control group (1.06% vs 2.43% counts/HPF, p<0.05) (FIG. 10).

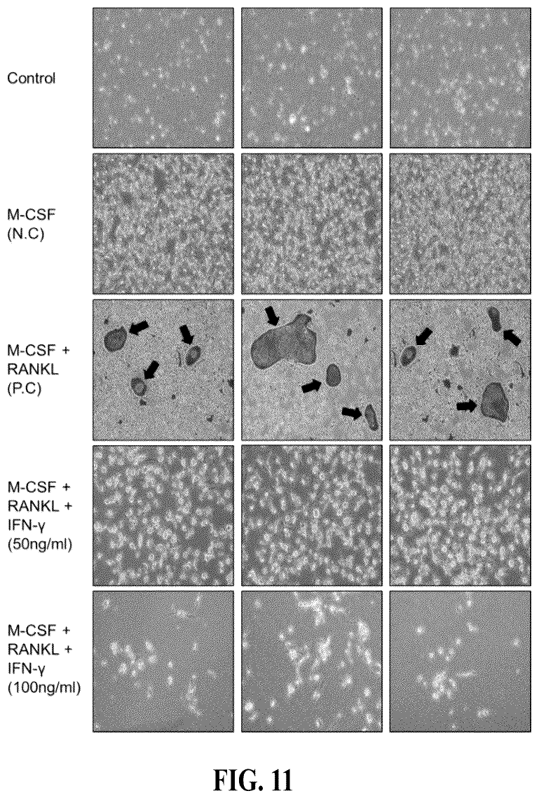

In addition, because it has been reported that IFN-γ was a key cytokine of T-helper type 1 (Th1) immunity and reduced osteoclastogenesis, whether IFN-γ inhibited osteoclastogenesis in vitro was evaluated. An IFN-γ treatment group completely inhibited osteoclastogenesis compared to a positive control group (FIG. 11).

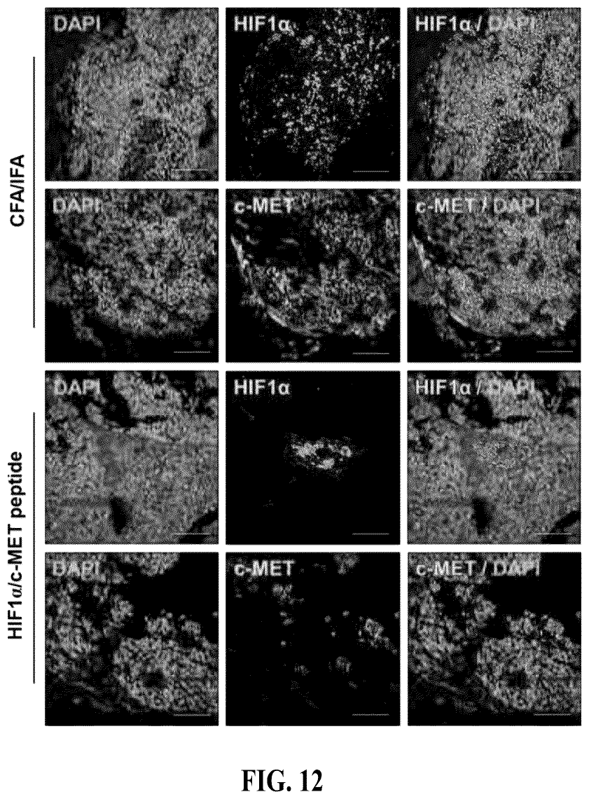

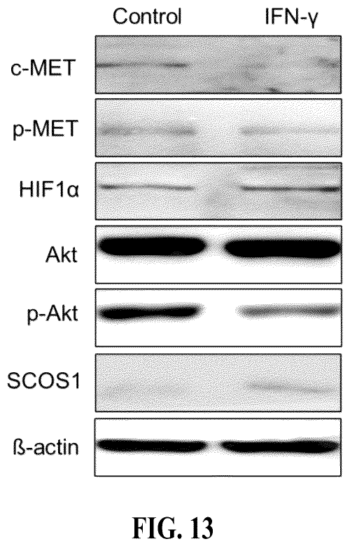

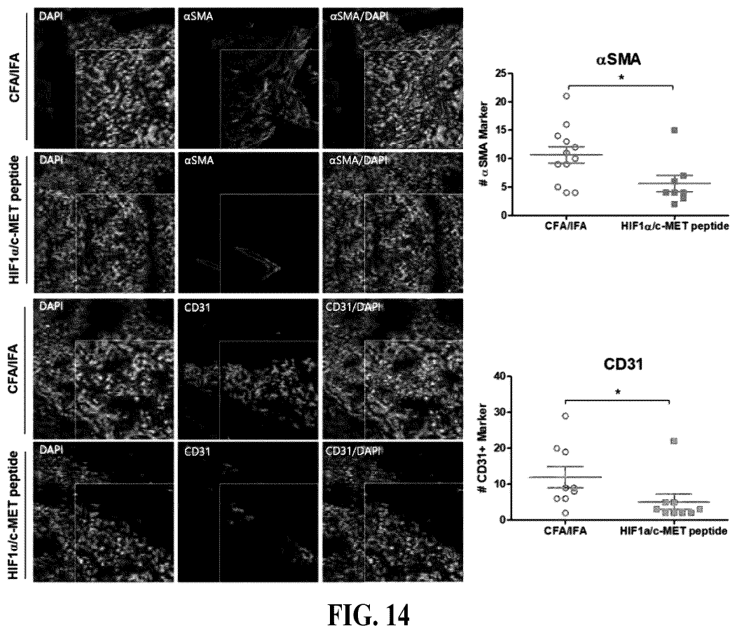

Further, because c-MET and HIF1α target proteins were decreased in immunized tumors compared to a control tumor, the effect of in vitro IFN-γ treatment on the expression of tumor proteins in M6-bone cells was examined (FIG. 12). Expression of c-MET and p-Akt was decreased in IFN-γ-treated M6-bone cells, whereas expression of SOCS1, known to regulate receptor tyrosine kinase signaling, was increased (FIG. 13). The expression of angiogenic markers α-SMA (mean; 10.6 vs. 5.6 counts, p<0.05) and CD31 (mean; 12 vs. 5.1 counts, p<0.05) was also significantly reduced in the vaccine group (FIG. 14).

From the result, it could be seen that the HIF1α and c-MET polypeptide vaccines induced more T cells into the bone microenvironment in an early bone metastasis progression model, stimulated Th cells to promote IFN-γ secretion, thereby inhibiting osteoclastogenesis and influencing the tumor microenvironment.

6. Confirmation of Tumor Growth Inhibition Through Combined Administration of HIF1α and c-MET Targeting Peptide Vaccines and Immune Checkpoint Inhibitor in Advanced Cancer Metastasis Model

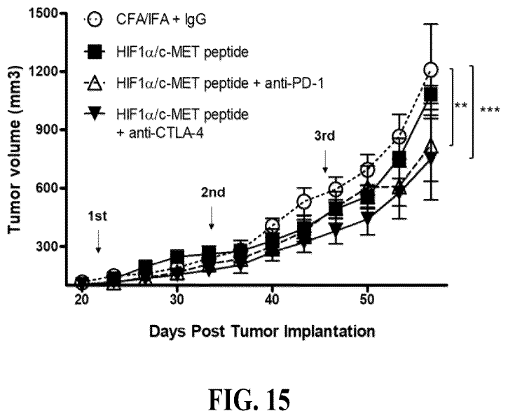

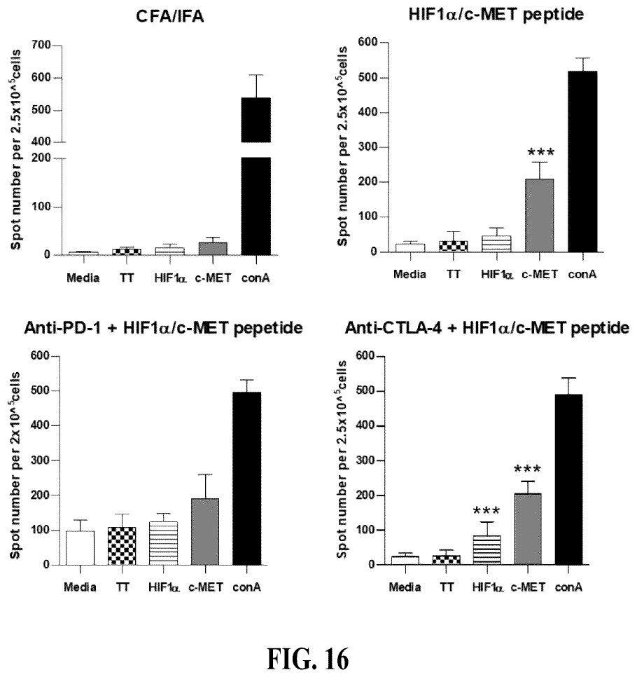

Since bone metastasis occurred according to cancer progression, vaccines alone may have a limited effect of treating metastatic bone tumors. Immune checkpoint proteins such as PD-1 and CTLA-4 were known to be highly expressed in TNBC patients. To enhance a therapeutic effect of the vaccine, the efficacy of combination therapy with the vaccine and the immune checkpoint inhibitor was evaluated in an advanced cancer mouse model. Unlike the early cancer model (FIG. 4), immunization with the vaccine did not have a significant therapeutic effect on the growth of implanted M6 tumors compared to the control group (vaccine vs control; 1081±46 vs 1208±234 mm³). In contrast, the tumor growth was effectively inhibited in a mouse group treated with a combination of the vaccine and an anti-PD1 or anti-CTLA4 antibody (818±184 mm³, p<0.005; 748±209 mm³, p<0.001) (FIG. 15). In addition, ELISPOT analysis showed that IFN-γ secreting Ag-specific T cells were most significantly induced in a combination of the HIF1α/c-MET polypeptide vaccine and anti-CTLA4 antibody treatment groups (FIG. 16).

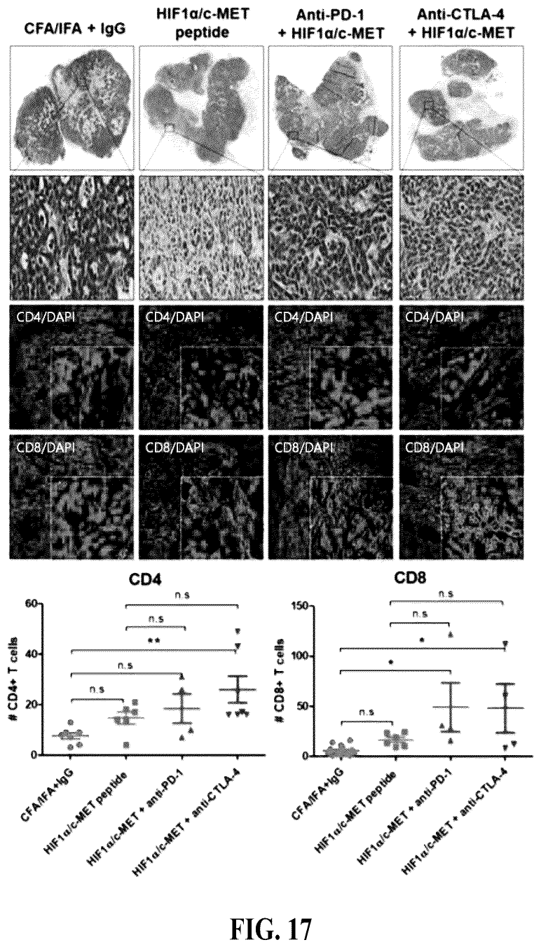

In addition, T cell infiltration into tumors was examined in relation to the immune microenvironment. Compared with the CFA/IFA control group, immune cell infiltration was significantly increased in a MET polypeptide+anti-CTLA-4 Ab combination treated group (mean; 7.5 vs 23.8 counts, p<0.05). Compared with the CFA/IFA control group, CD8 T cell infiltration was significantly increased in both an HIF1α/c-MET polypeptide+anti-PD-1 Ab combination treated group (mean; 5.6 vs 41.2 counts, p<0.005) and an HIF1α/c-MET polypeptide+CTLA-4 Ab combination treated group (mean; 5.6 vs 40 counts, p<0.05) (FIG. 17).

From the results, it may be seen that combination therapy with the HIF1α/c-MET polypeptide vaccine and the immune checkpoint inhibitor is more effective in inhibiting tumor growth in an advanced cancer model as measured by increased Ag-specific T cell immune responses and T cell infiltration.

7. Confirmation of Bone Metastasis Inhibition Through Combined Administration of HIF1α and c-MET Targeting Peptide Vaccines and Immune Checkpoint Inhibitor in Advanced Cancer Metastasis Model

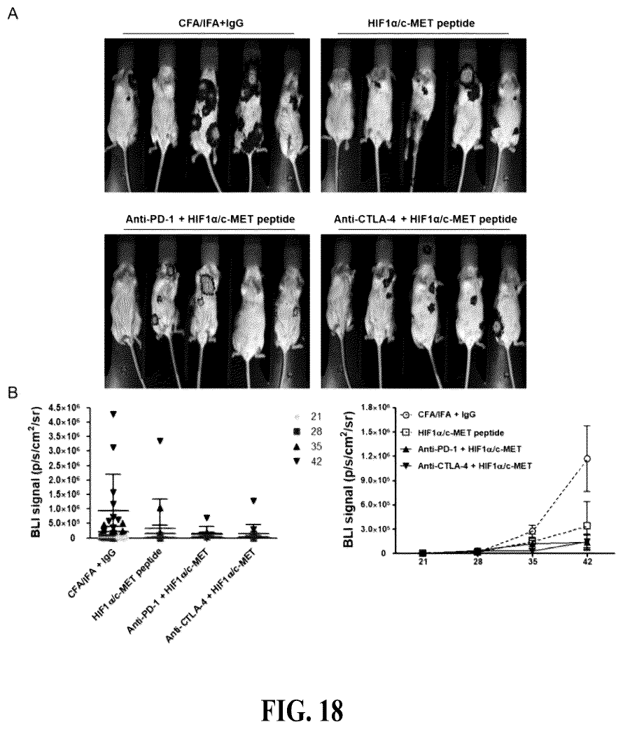

Like the previous results, the effect of combination therapy with the vaccines and the immune checkpoint inhibitor was evaluated by constructing an advanced systemic metastasis model. Mice were randomly assigned to receive each treatment and the bone metastasis inhibition was measured every week using bioluminescence imaging. The mean luciferase signal evaluated 42 days after intracardiac injection was measured to be lower in the HIF1α/c-MET polypeptide+anti-PD-1 Ab combination group and the HIF1α/c-MET polypeptide+anti-CTLA-4 Ab combination group compared to a CFA/IFA or HIF1α/c-MET polypeptide alone group (FIG. 18).

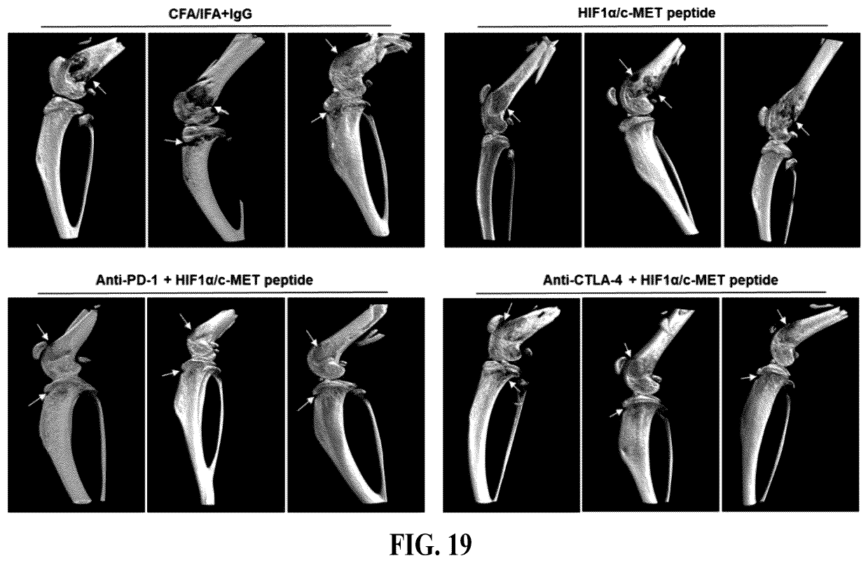

Subsequently, the degree of bone destruction in each group was evaluated using micro-CT analysis. Three-dimensional reconstructed micro-CT images of bones in the representative CFA/IFA and HIF1α/c-MET polypeptide-alone groups showed severe bone destruction. In contrast, the HIF1α/c-MET polypeptide+anti-PD-1 Ab combination group and the HIF1α/c-MET polypeptide+anti-CTLA-4 Ab combination group showed limited bone destruction (FIG. 19).

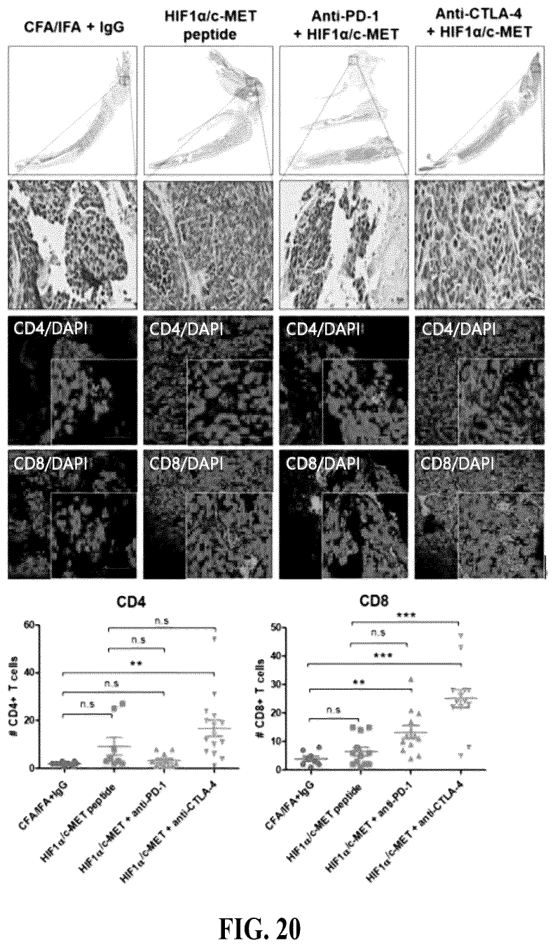

In addition, T cell infiltration in metastatic bone tumors was examined. It was confirmed that as compared with the CFA/IFA group, more CD8 T cells (mean; 7 vs 12.2 counts, p<0.005) were infiltrated in the HIF1α/c-MET polypeptide+anti-PD-1 Ab combination treated group, but CD4 T cells were not infiltrated. In the anti-CTLA-4 Ab combination group, as compared with the CFA/IFA group and the HIF1α/c-MET polypeptide alone group, CD4 (mean; 1.8 vs 16.8 counts, p<0.005) and CD8 T cells (mean; 7 vs 25.2 counts, p<0.001), (mean; 6.5 vs 25.2 counts, p<0.001) were significantly more densely infiltrated (FIG. 20).

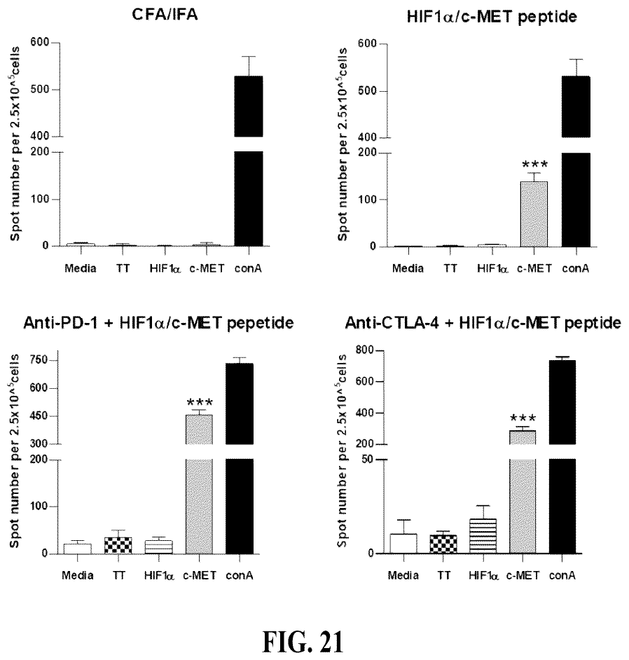

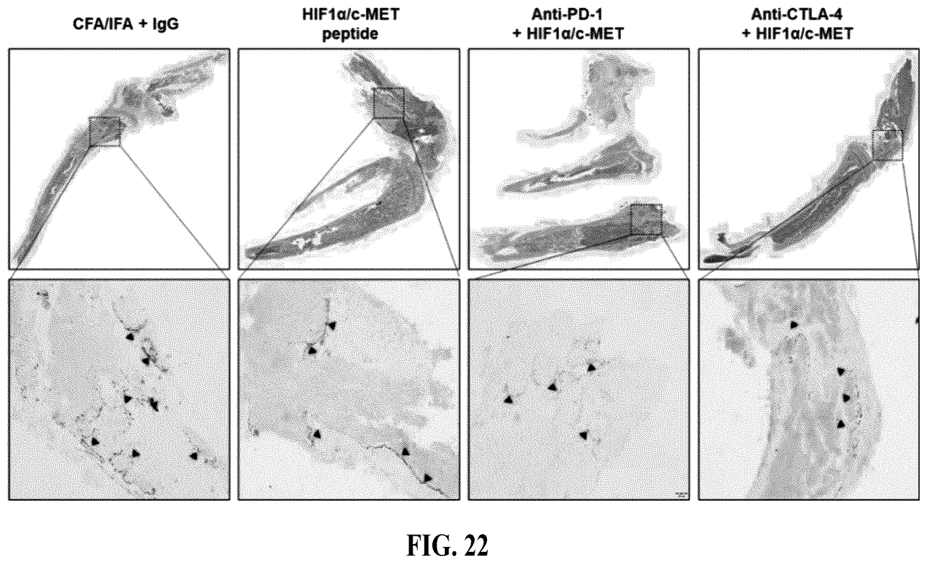

In addition, IFN-γ ELISPOT analysis showed that Ag-specific T cells were further expanded when stimulated with the c-MET peptide in the combination therapy group than in the HIF1α/c-MET polypeptide alone group (FIG. 21). In addition, subsequent analysis using H&E staining and TRAP staining showed that the number and activity of osteoclasts in metastatic bone lesions in the combination therapy group were significantly reduced compared to the CFA/IFA and HIF1α/c-MET polypeptide alone groups (FIG. 22).

From the results, it may be seen that in the advanced bone metastasis, combination therapy with the HIF1α/c-MET polypeptide vaccine and the immune checkpoint inhibitor inhibits the growth of metastatic bone tumors and further reduces osteoclast differentiation by increasing Ag-specific T cell immune responses and T cell infiltration in the bone microenvironment.

8. Confirmation of Anticancer Effects of HIF1α and c-MET Polypeptides in Chemotherapy-Resistant Cancer Type

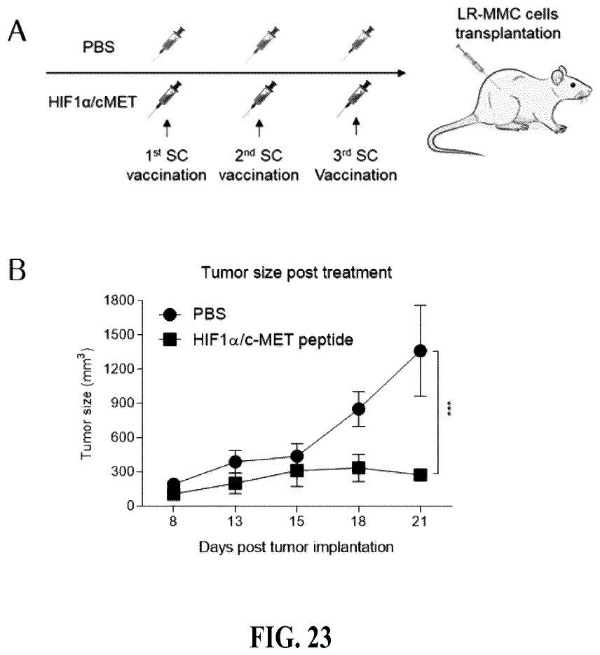

Whether the growth of therapy-resistant cell lines could be inhibited by tumor vaccines using a lapatinib-resistant cell line was intended to be confirmed. The HIF1α/c-MET polypeptide was administered three times to MMTV-neu transgenic mice and inoculated with the lapatinib-resistant cell line (FIG. 23A). On day 35 after vaccination, the mice were sacrificed and tumor sizes were measured, and as a result, it was confirmed that the tumor growth of mice administered with the HIF1α/c-MET vaccine was significantly inhibited compared to the control group (FIG. 23B).

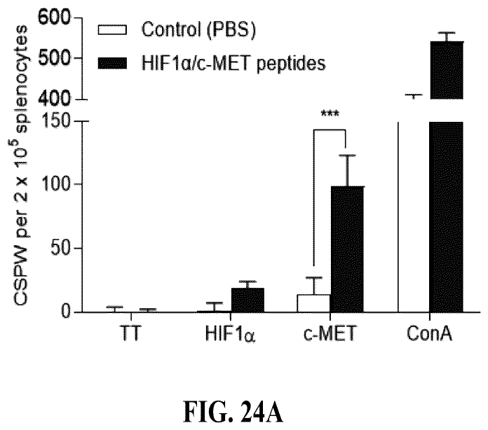

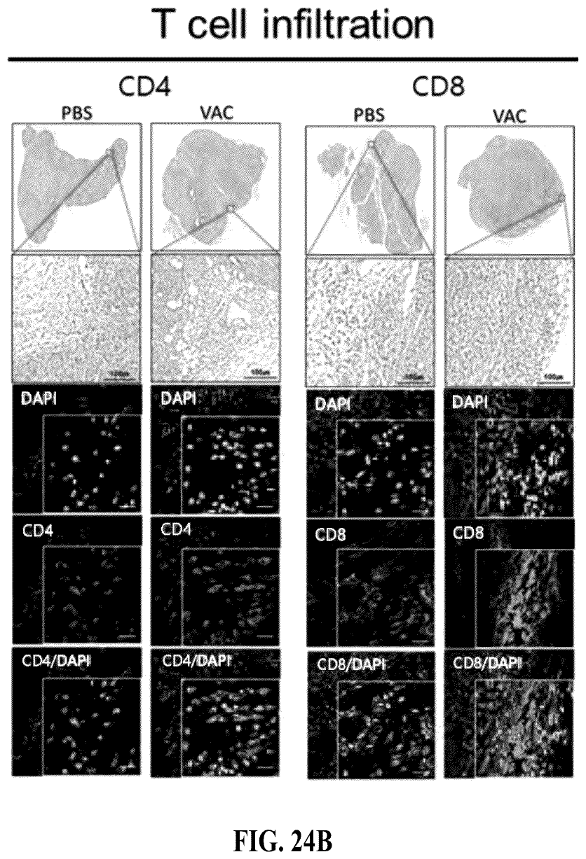

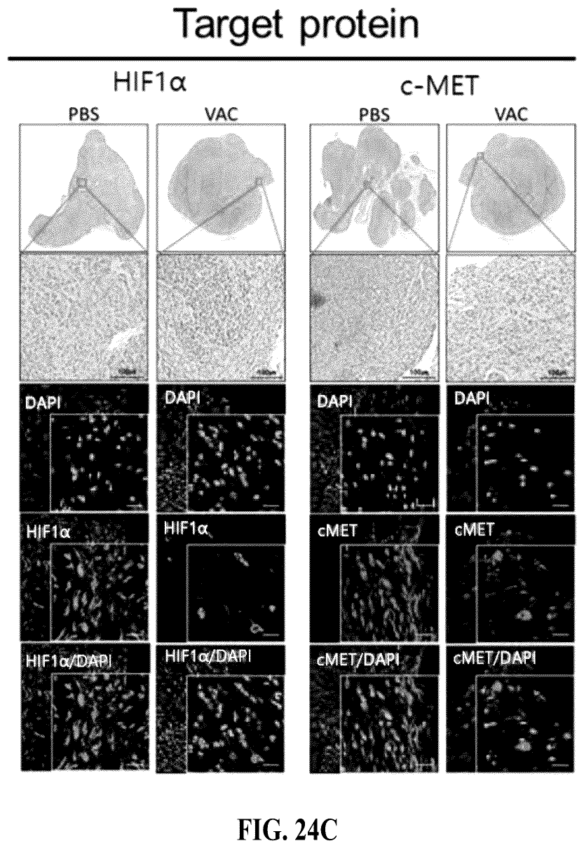

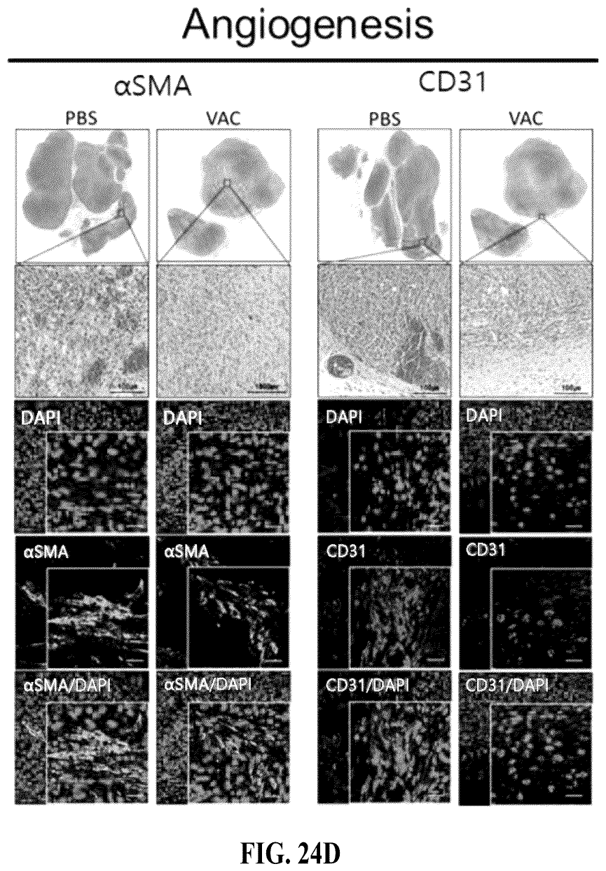

In addition, IFN-γ ELISPOT analysis showed that Ag-specific T cells were further expanded upon HIF1α/c-MET polypeptide stimulation (FIG. 24A). In addition, subsequent analysis using H&E staining and immunofluorescence staining showed that the HIF1α/c-MET polypeptide was administered to increase T cells infiltrated into the tumors (FIG. 24B), reduce the expression of the target protein (FIG. 24C), and reduce the expression of angiogenic marker proteins (FIG. 24D).

9. Confirmation of Metastatic Cancer Inhibition Effect of HIF1α and c-MET Polypeptides

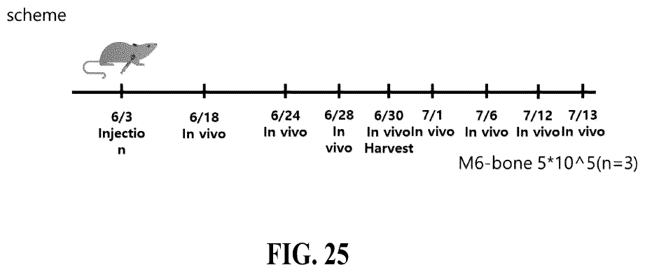

Next, it was intended to determine whether HIF1α and c-MET polypeptides could prevent systemic metastatic cancer in addition to the effect of preventing bone metastatic cancer. The experimental schedule was as shown in FIG. 25.

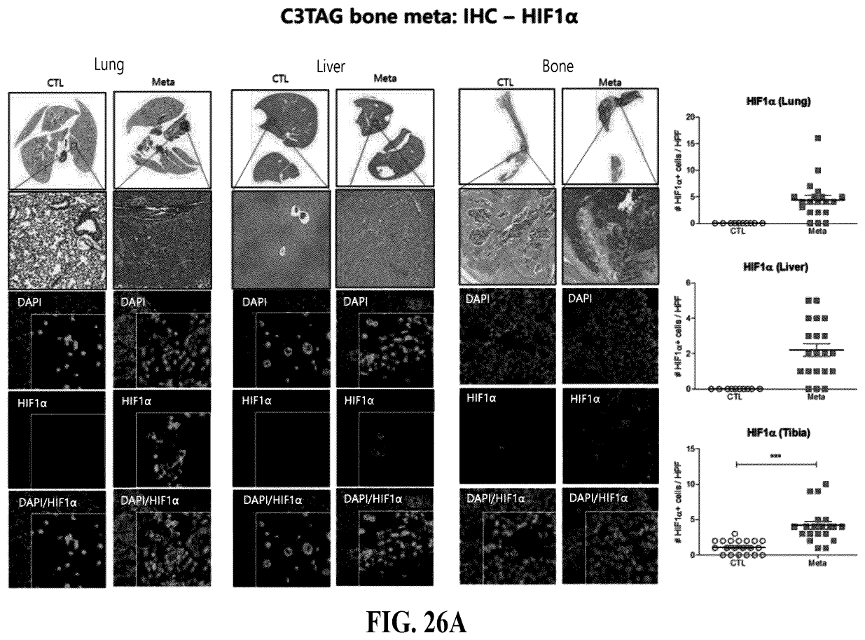

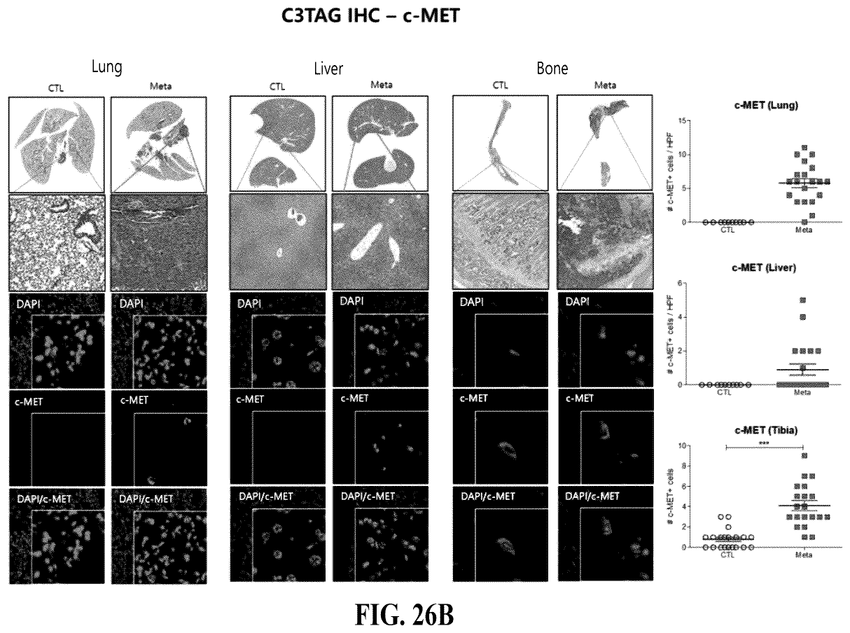

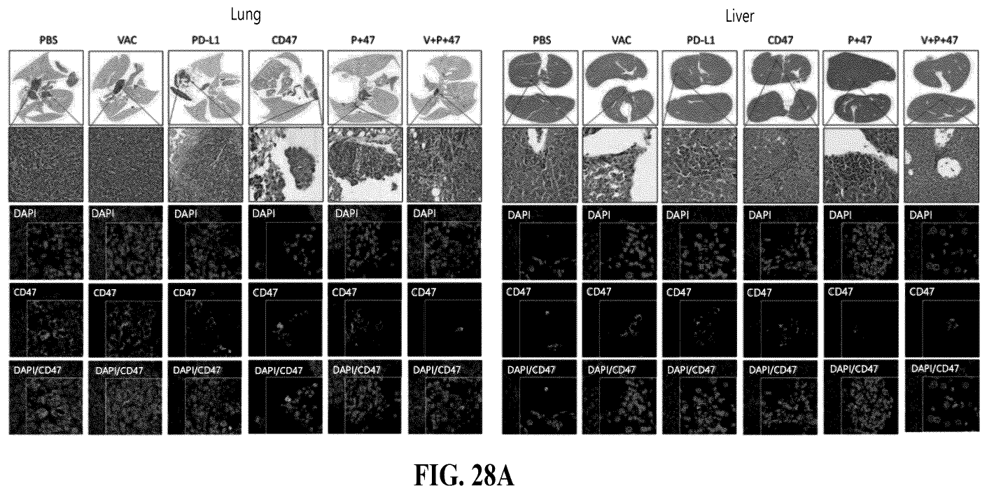

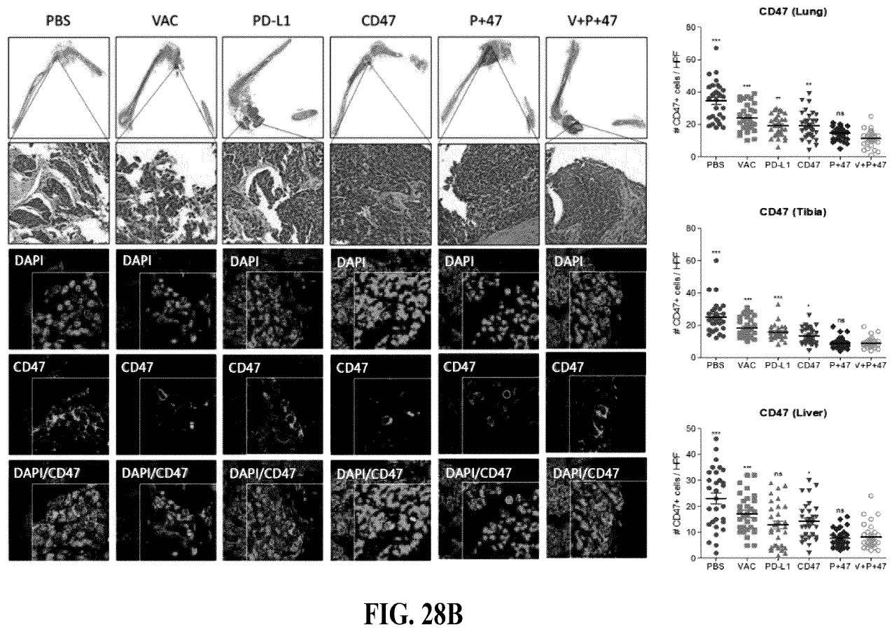

First, it was confirmed whether systemic metastatic cancer was induced by administration of the M6-bone cell line. It was confirmed that when the M6-bone cell line, which mainly caused bone metastasis, was injected into the heart of mice, the number of cells expressing c-MET and HIF1α increased in the lung, liver, and bone (FIGS. 26A and 26B), and the cancer cells overexpressed CD47, an immune checkpoint protein (FIGS. 28A and 28B).

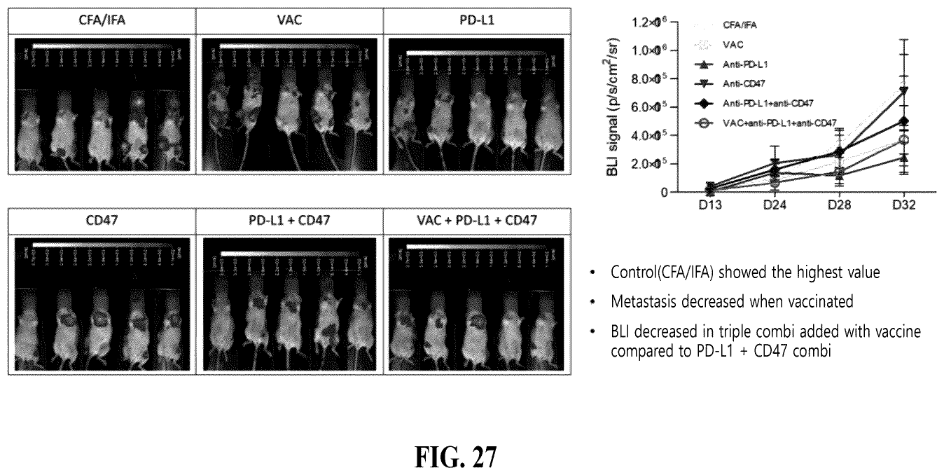

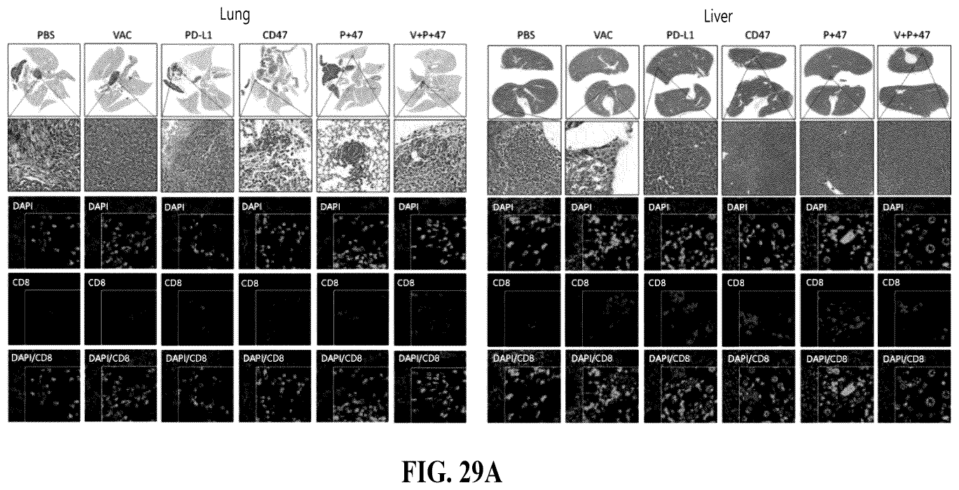

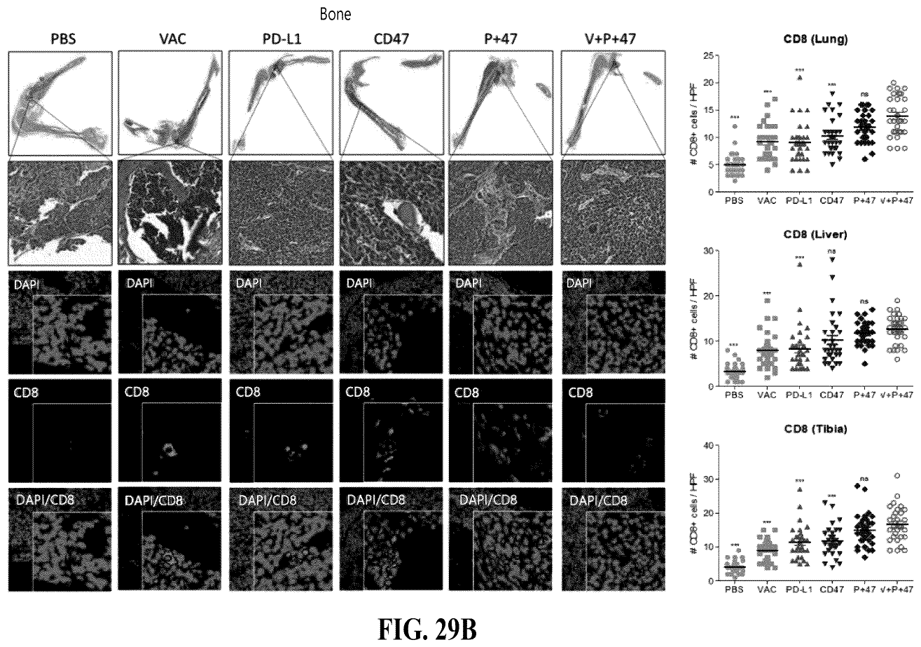

In addition, when c-MET, HIF1α tumor vaccine alone (VAC) and combination with the immune checkpoint inhibitor were administered to each mouse three times, and then cancer cells were injected into the heart, compared to the control group, metastatic cancer was reduced in each treatment group, and particularly, the combined administration group showed the most excellent effect in preventing metastatic cancer (FIGS. 27 and 29).

As described above, although the embodiments have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other embodiments, and equivalents to the appended claims fall within the scope of the claims to be described below.

1. A pharmaceutical composition for preventing or treating cancer comprising an epitope peptide of HIF1α and an epitope peptide of c-Met as an active ingredient.

2. The pharmaceutical composition of claim 1, wherein the epitope peptide of c-Met comprises at least one amino acid sequence selected from the group consisting of amino acid sequences represented by SEQ ID NOs: 1 and 2.

3. The pharmaceutical composition of claim 1, wherein the epitope peptide of HIF1α comprises at least one amino acid sequence selected from the group consisting of amino acid sequences represented by SEQ ID NOs: 3 to 5.

4. The pharmaceutical composition of claim 1, wherein the epitope peptide of HIF1α and the epitope peptide of c-Met are fused to be one polypeptide.

5. The pharmaceutical composition of claim 4, wherein the polypeptide comprises an amino acid sequence represented by SEQ ID NO: 6.

6. The pharmaceutical composition of claim 1, wherein the pharmaceutical composition is for co-administration with an immune checkpoint inhibitor.

7. The pharmaceutical composition of claim 1, further comprising:
an immune checkpoint inhibitor.

8. The pharmaceutical composition of claim 6 or 7, wherein the immune checkpoint inhibitor blocks signaling of at least one immune checkpoint protein selected from the group consisting of CTLA-4, PD-1, PD-L1, PD-L2, LAG-3, BTLA, B7H3, B7H4, TIM3, KIR, TIGIT, CD47, VISTA and A2aR.

9. The pharmaceutical composition of claim 6 or 7, wherein the immune checkpoint inhibitor is an antibody targeting at least one immune checkpoint protein selected from the group consisting of CTLA-4, PD-1, PD-L1, PD-L2, LAG-3, BTLA, B7H3, B7H4, TIM3, KIR, TIGIT, CD47, VISTA and A2aR.

10. The pharmaceutical composition of claim 9, wherein the antibody is a monoclonal antibody.

11. The pharmaceutical composition of claim 1, wherein the cancer is at least one selected from the group consisting of breast cancer, lung cancer, stomach cancer, head and neck cancer, kidney cancer, liver cancer, prostate cancer, and thyroid cancer.

12. The pharmaceutical composition of claim 1, wherein the cancer is metastatic cancer.

13. The pharmaceutical composition of claim 12, wherein the metastatic cancer is metastatic bone tumor.


SEQUENCE LISTING (6 sequences)

SEQ ID NO: 1 - protein, 15 aa, synthetic construct
  REGION 1..15: note = cMet epitope(P275)
HTRIIRFCSINSGLH

SEQ ID NO: 2 - protein, 15 aa, synthetic construct
  REGION 1..15: note = cMet epitope(P314)
FNILQAAYVSKPGAQ

SEQ ID NO: 3 - protein, 16 aa, synthetic construct
  REGION 1..16: note = HIF1alpha epitope(p38-53)
YELAHQLPLPHNVSSH

SEQ ID NO: 4 - protein, 23 aa, synthetic construct
  REGION 1..23: note = HIF1alpha epitope(p60-82)
MRLTISYLRVRKLLDAGDLDIED

SEQ ID NO: 5 - protein, 25 aa, synthetic construct
  REGION 1..25: note = HIF1alpha epitope(p93-117)
LKALDGFVMVLTDDGDMIYISDNVN

SEQ ID NO: 6 - protein, 94 aa, synthetic construct
  REGION 1..94: note = HIF1alpha/cMet polypeptide
HTRIIRFCSINSGLHFNILQAAYVSKPGAQYELAHQLPLPHNVSSHMRLTISYLRVRKLLDAGDLDIEDLKALDGFVMVLTDDGDMIYISDNVN